SEQUENCE LISTING (20 sequences)

SEQ ID NO: 1 - protein, 269 aa, Potato virus A
AETLDASEALAQKSEGRKKEEESNSSKAVAVKDKDVDLGTAGTHSVPRLKSMTSKLTLPMLKGKSVVNLDHLLSYKPKQVDLSNARATHEQFQNWYDGVMASYELEESSMEIILNGFMVWCIENGTSPDINGVWTMMDNEEQVSYPLKPMLDHAKPSLRQIMRHFSALAEAYIEMRSREKPYMPRYGLQRNLRDQSLARYAFDFYEITATTPIRAKEAHLQMKAAALKNSNTNMFGLDGNVTTSEEDTERHTATDVNRNMHHLLGVKGV

SEQ ID NO: 2 - DNA, 809 bp, Potato virus A
agccgaaactcttgatgcaagcgaagcactagcgcagaaatctgaaggtaggaagaaagaaagagaaagcaacagtagcaaagctgtagccgtgaaggacaaagatgtcgatttaggtactgctgggactcattcagtaccacgcttaaaatcaatgacatcaaaactgacactaccaatgctcaaaggtaagagtgtcgttaacctagatcacttgctatcttacaaaccaaagcaagtagacttgtcaaatgctagagccacccacgaacaattccaaaactggtatgatggcgttatggcaagttatgagttagaggaatcaagcatggagatcattctaaatggcttcatggtatggtgcattgagaatggaacctctccagacattaatggagtttggaccatgatggataatgaggaacaagtgtcatatccattaaaacccatgcttgaccatgcaaagccttctttaaggcaaattatgagacatttcagcgcactcgcagaggcgtacattgagatgagaagtcgtgagaaaccatacatgcccaggtatggtcttcaacgcaacctgagagatcaaagtttggcaaggtatgcttttgatttctatgagatcactgcaaccactccgatcagagccaaagaggcgcatctgcagatgaaagcagcagcgctgaagaattcgaacactaatatgtttggactggacggaaatgtcacaacttcagaagaggacacagaaaggcacacagcaacggatgttaatcgcaacatgcatcaccttttaggcgtgaagggggtgt

SEQ ID NO: 3 - protein, 285 aa, Artificial Sequence
  melanoma sequence inserted into PVA by mutagenesis
MHHHHHHGSIMDQVPFSVALAQKSEGRKKEEESNSSKAVAVKDKDVDLGTAGTHSVPRLKSMTSKLTLPMLKGKSVVNLDHLLSYKPKQVDLSNARATHEQFQNWYDGVMASYELEESSMEIILNGFMVWCIENGTSPDINGVWTMMDNEEQVSYPLKPMLDHAKPSLRQIMRHFSALAEAYIEMRSREKPYMPRYGLQRNLRDQSLARYAFDFYEITATTPIRAKEAHLQMKAAALKNSNTNMFGLDGNVTTSEEDTERHTATDVNRNMHHLLGVKGVVALLPS

SEQ ID NO: 4 - DNA, 853 bp, Artificial Sequence
  melanoma sequence inserted into PVA by mutagenesis
atgcaccatcaccatcaccatggatccatcatggaccaggtgccgttcgcactagcgcagaaatctgaaggtaggaagaaagaaagagaaagcaacagtagcaaagctgtagccgtgaaggacaaagatgtcgatttaggtactgctgggactcattcagtaccacgcttaaaatcaatgacatcaaaactgacactaccaatgctcaaaggtaagagtgtcgttaacctagatcacttgctatcttacaaaccaaagcaagtagacttgtcaaatgctagagccacccacgaacaattccaaaactggtatgatggcgttatggcaagttatgagttagaggaatcaagcatggagatcattctaaatggcttcatggtatggtgcattgagaatggaacctctccagacattaatggagtttggaccatgatggataatgaggaacaagtgtcatatccattaaaacccatgcttgaccatgcaaagccttctttaaggcaaattatgagacatttcagcgcactcgcagaggcgtacattgagatgagaagtcgtgagaaaccatacatgcccaggtatggtcttcaacgcaacctgagagatcaaagtttggcaaggtatgcttttgatttctatgagatcactgcaaccactccgatcagagccaaagaggcgcatctgcagatgaaagcagcagcgctgaagaattcgaacactaatatgtttggactggacggaaatgtcacaacttcagaagaggacacagaaaggcacacagcaacggatgttaatcgcaacatgcatcaccttttaggcgtgaagggggtgtgtcgacctgcagccaagctaa

SEQ ID NO: 5 - protein, 293 aa, Artificial Sequence
  melanoma sequence inserted into PVA by mutagenesis
MHHHHHHGSAETLDASEALAQKSEGRKKEEESNSSKAVAVKDKDVDLGTAGTHSVPRLKSMTSKLTLPMLKGKSVVNLDHLLSYKPKQVDLSNARATHEQFQNWYDGVMASYELEESSMEIILNGFMVWCIENGTSPDINGVWTMMDNEEQVSYPLKPMLDHAKPSLRQIMRHFSALAEAYIEMRSREKPYMPRYGLQRNLRDQSLARYAFDFYEITATTPIRAKEAHLQMKAAALKNSNTNMFGLDGNVTTSEEDTERHTATDVNRNMHHLLGVKGVIMDQVPFSVVALLPS

SEQ ID NO: 6 - DNA, 883 bp, Artificial Sequence
  melanoma sequence inserted into PVA by mutagenesis
atgcaccatcaccatcaccatggatccgccgaaactcttgatgcaagcgaagcactagcgcagaaatctgaaggtaggaagaaagaaagagaaagcaacagtagcaaagctgtagccgtgaaggacaaagatgtcgatttaggtactgctgggactcattcagtaccacgcttaaaatcaatgacatcaaaactgacactaccaatgctcaaaggtaagagtgtcgttaacctagatcacttgctatcttacaaaccaaagcaagtagacttgtcaaatgctagagccacccacgaacaattccaaaactggtatgatggcgttatggcaagttatgagttagaggaatcaagcatggagatcattctaaatggcttcatggtatggtgcattgagaatggaacctctccagacattaatggagtttggaccatgatggataatgaggaacaagtgtcatatccattaaaacccatgcttgaccatgcaaagccttctttaaggcaaattatgagacatttcagcgcactcgcagaggcgtacattgagatgagaagtcgtgagaaaccatacatgcccaggtatggtcttcaacgcaacctgagagatcaaagtttggcaaggtatgcttttgatttctatgagatcactgcaaccactccgatcagagccaaagaggcgcatctgcagatgaaagcagcagcgctgaagaattcgaacactaatatgtttggactggacggaaatgtcacaacttcagaagaggacacagaaaggcacacagcaacggatgttaatcgcaacatgcatcaccttttaggcgtgaagggggtgtatcatggaccaggtgccgttctccgtcgtcgacctgcagccagcttaa

SEQ ID NO: 7 - protein, 275 aa, Artificial Sequence
  melanoma sequence inserted into PVA by mutagenesis
MGSIMDQVPFSVALAQKSEGRKKEEESNSSKAVAVKDKDVDLGTAGTHSVPRLKSMTSKLTLPMLKGKSVVNLDHLLSYKPKQVDLSNARATHEQFQNWYDGVMASYELEESSMEIILNGFMVWCIENGTSPDINGVWTMMDNEEQVSYPLKPMLDHAKPSLRQIMRHFSALAEAYIEMRSREKPYMPRYGLQRNLRDQSLARYAFDFYEITATTPIRAKEAHLQMKAAALKNSNTNMFGLDGNVTTSEEDTERHTATDVNRNMHHLLGVKGVLQ

SEQ ID NO: 8 - DNA, 829 bp, Artificial Sequence
  melanoma sequence inserted into PVA by mutagenesis
ggatccatgatcatggaccaggtgccgttctccgtcgcactagcgcagaaatctgaaggtaggaagaaagaaagagaaagcaacagtagcaaagctgtagccgtgaaggacaaagatgtcgatttaggtactgctgggactcattcagtaccacgcttaaaatcaatgacatcaaaactgacactaccaatgctcaaaggtaagagtgtcgttaacctagatcacttgctatcttacaaaccaaagcaagtagacttgtcaaatgctagagccacccacgaacaattccaaaactggtatgatggcgttatggcaagttatgagttagaggaatcaagcatggagatcattctaaatggcttcatggtatggtgcattgagaatggaacctctccagacattaatggagtttggaccatgatggataatgaggaacaagtgtcatatccattaaaacccatgcttgaccatgcaaagccttctttaaggcaaattatgagacatttcagcgcactcgcagaggcgtacattgagatgagaagtcgtgagaaaccatacatgcccaggtatggtcttcaacgcaacctgagagatcaaagtttggcaaggtatgcttttgatttctatgagatcactgcaaccactccgatcagagccaaagaggcgcatctgcagatgaaagcagcagcgctgaagaattcgaacactaatatgtttggactggacggaaatgtcacaacttcagaagaggacacagaaaggcacacagcaacggatgttaatcgcaacatgcatcaccttttaggcgtgaagggggtgtctcgagtaa

SEQ ID NO: 9 - protein, 283 aa, Artificial Sequence
  melanoma sequence inserted into PVA by mutagenesis
MGSAETLDASEALAQKSEGRKKEEESNSSKAVAVKDKDVDLGTAGTHSVPRLKSMTSKLTLPMLKGKSVVNLDHLLSYKPKQVDLSNARATHEQFQNWYDGVMASYELEESSMEIILNGFMVWCIENGTSPDINGVWTMMDNEEQVSYPLKPMLDHAKPSLRQIMRHFSALAEAYIEMRSREKPYMPRYGLQRNLRDQSLARYAFDFYEITATTPIRAKEAHLQMKAAALKNSNTNMFGLDGNVTTSEEDTERHTATDVNRNMHHLLGVKGVIMDQVPFSVLQ

SEQ ID NO: 10 - DNA, 853 bp, Artificial Sequence
  melanoma sequence inserted into PVA by mutagenesis
atgggatccgccgaaactcttgatgcaagcgaagcactagcgcagaaatctgaaggtaggaagaaagaaagagaaagcaacagtagcaaagctgtagccgtgaaggacaaagatgtcgatttaggtactgctgggactcattcagtaccacgcttaaaatcaatgacatcaaaactgacactaccaatgctcaaaggtaagagtgtcgttaacctagatcacttgctatcttacaaaccaaagcaagtagacttgtcaaatgctagagccacccacgaacaattccaaaactggtatgatggcgttatggcaagttatgagttagaggaatcaagcatggagatcattctaaatggcttcatggtatggtgcattgagaatggaacctctccagacattaatggagtttggaccatgatggataatgaggaacaagtgtcatatccattaaaacccatgcttgaccatgcaaagccttctttaaggcaaattatgagacatttcagcgcactcgcagaggcgtacattgagatgagaagtcgtgagaaaccatacatgcccaggtatggtcttcaacgcaacctgagagatcaaagtttggcaaggtatgcttttgatttctatgagatcactgcaaccactccgatcagagccaaagaggcgcatctgcagatgaaagcagcagcgctgaagaattcgaacactaatatgtttggactggacggaaatgtcacaacttcagaagaggacacagaaaggcacacagcaacggatgttaatcgcaacatgcatcaccttttaggcgtgaagggggtgtatcatggaccaggtgccgttctccgtcctcgagtaa

SEQ ID NO: 11 - protein, 9 aa, Homo sapiens
IMDQVPFSV

SEQ ID NO: 12 - DNA, 27 bp, Homo sapiens
atcatggaccaggtgccgttctccgtc

SEQ ID NO: 13 - DNA, 809 bp, Potato virus A
agccgaaactcttgatgcaagcgaagcactagcgcagaaatctgaaggtaggaagaaagaaagagaaagcaacagtagcaaagctgtagccgtgaaggacaaagatgtcgatttaggtactgctgggactcattcagtaccacgcttaaaatcaatgacatcaaaactgacactaccaatgctcaaaggtaagagtgtcgttaacctagatcacttgctatcttacaaaccaaagcaagtagacttgtcaaatgctagagccacccacgaacaattccaaaactggtatgatggcgttatggcaagttatgagttagaggaatcaagcatggagatcattctaaatggcttcatggtatggtgcattgagaatggaacctctccagacattaatggagtttggaccatgatggataatgaggaacaagtgtcatatccattaaaacccatgcttgaccatgcaaagccttctttaaggcaaattatgagacatttcagcgcactcgcagaggcgtacattgagatgagaagtcgtgagaaaccatacatgcccgggtatggtcttcaacgcaacctgagagatcaaagtttggcaaggtatgcttttgatttctatgagatcactgcaaccactccgatcggagccaaagaggcgcatctgcagatgaaagcagcagcgctgaagaattcgaacactaatatgtttggactggacggaaatgtcacaacttcagaagaggacacagaaaggcacacagcaacggatgttaatcgcaacatgcatcaccttttaggcgtgaagggggtgt

SEQ ID NO: 14 - protein, 269 aa, Potato virus A
AETLDASEALAQKSEGRKKEEESNSSKAVAVKDKDVDLGTAGTHSVPRLKSMTSKLTLPMLKGKSVVNLDHLLSYKPKQVDLSNARATHEQFQNWYDGVMASYELEESSMEIILNGFMVWCIENGTSPDINGVWTMMDNEEQVSYPLKPMLDHAKPSLRQIMRHFSALAEAYIEMRSREKPYMPGYGLQRNLRDQSLARYAFDFYEITATTPIRAKEAHLQMKAAALKNSNTNMFGLDGNVTTSEEDTERHTATDVNRNMHHLLGVKGV

SEQ ID NO: 15 - DNA, 808 bp, Potato virus A
agccgaaactcttgatgcaagcgaagcactagcgcagaaatctgaaggtaggaagaaagaaagagaaagcaacagtagcaaagctgtagccgtgaaggacaaagatgtcgtttaggtactgctgggactcattcagtaccacgcttaaaatcaatgacatcaaaactgacactaccaatgctcaaaggtaagagtgtcgttaacctagatcacttgctatcttacaaaccaaagcaagtagacttgtcaaatgctagagccacccacgaacaattccaaaactggtatgatggcgttatggcaagttatgagttagaggaatcaagcatggagatcattctaaatggcttcatggtatggtgcattgagaatggaacctctccagacattaatggagtttggaccatgatggataatgaggaacaagtgtcatatccattaaaacccatgcttgaccatgcaaagccttctttaaggcaaattatgagacatttcagcgcactcgcagaggcgtacattgagatgagaagtcgtgagaaaccatacatgcccaggtatggtcttcaacgcaacctgagagatcaaagtttggcaaggtatgcttttgatttctatgagatcactgcaaccactccgatcggagccaaagaggcgcatctgcagatgaaagcagcagcgctgaagaattcgaacactaatatgtttggactggacggaaatgtcacaacttcagaagaggacacagaaaggcacacagcaacggatgttaatcgcaacatgcatcaccttttaggcgtgaagggggtgt

SEQ ID NO: 16 - protein, 269 aa, Potato virus A
AETLDASEALAQKSEGRKKEEESNSSKAVAVKDKDVDLGTAGTHSVPRLKSMTSKLTLPMLKGKSVVNLDHLLSYKPKQVDLSNARATHEQFQNWYDGVMASYELEESSMEIILNGFMVWCIENGTSPDINGVWTMMDNEEQVSYPLKPMLDHAKPSLRQIMRHFSALAEAYIEMRSREKPYMPRYGLQRNLRDQSLARYAFDFYEITATTPIGAKEAHLQMKAAALKNSNTNMFGLDGNVTTSEEDTERHTATDVNRNMHHLLGVKGV

SEQ ID NO: 17 - DNA, 809 bp, Potato virus A
agccgaaactcttgatgcaagcgaagcactagcgcagaaatctgaaggtaggaagaaagaaagagaaagcaacagtagcaaagctgtagccgtgaaggacaaagatgtcgatttaggtactgctgggactcattcagtaccacgcttaaaatcaatgacatcaaaactgacactaccaatgctcaaaggtaagagtgtcgttaacctagatcacttgctatcttacaaaccaaagcaagtagacttgtcaaatgctagagccacccacgaacaattccaaaactggtatgatggcgttatggcaagttatgagttagaggaatcaagcatggagatcattctaaatggcttcatggtatggtgcattgagaatggaacctctccagacattaatggagtttggaccatgatggataatgaggaacaagtgtcatatccattaaaacccatgcttgaccatgcaaagccttctttaaggcaaattatgagacatttcagcgcactcgcagaggcgtacattgagatgagaagtcgtgagaaaccatacatgcccaggtatggtcttcaacgcaacctgagagatcaaagtttggcaaggtatgcttttgatttctatgagatcactgcaaccactccgatcagagccaaagaggcgcatctgctgatgaaagcagcagcgctgaagaattcgaacactaatatgtttggactggacggaaatgtcacaacttcagaagaggacacagaaaggcacacagcaacggatgttaatcgcaacatgcatcaccttttaggcgtgaagggggtgt

SEQ ID NO: 18 - protein, 269 aa, Potato virus A
AETLDASEALAQKSEGRKKEEESNSSKAVAVKDKDVDLGTAGTHSVPRLKSMTSKLTLPMLKGKSVVNLDHLLSYKPKQVDLSNARATHEQFQNWYDGVMASYELEESSMEIILNGFMVWCIENGTSPDINGVWTMMDNEEQVSYPLKPMLDHAKPSLRQIMRHFSALAEAYIEMRSREKPYMPRYGLQRNLRDQSLARYAFDFYEITATTPIRAKEAHLLMKAAALKNSNTNMFGLDGNVTTSEEDTERHTATDVNRNMHHLLGVKGV

SEQ ID NO: 19 - DNA, 807 bp, Potato virus A
agccgaaactcttgatgcaagcgaagcactagcgcagaaatctgaaggtaggaagaaagaaagagaaagcaacagtagcaaagctgtagccgtgaaggacaaagatgtcgatttaggtactgctgggactcattcagtaccacgcttaaaatcaatgacatcaaaactgacactaccaatgctcaaaggtaagagtgtcgttaacctagatcacttgctatcttacaaaccaaagcaagtagacttgtcaaatgctagagccacccacgaacaattccaaaactggtatgatggcgttatggcaagttatgagttagaggaatcaagcatggagatcattctaaatggcttcatggtatggtgcattgagaatggaacctctccagacattaatggagtttggaccatgatggataatgaggaacaagtgtcatatccattaaaacccatgcttgaccatgcaaagccttctttaaggcaaattatgagacatttcagcgcactcgcagaggcgtacattgagatgagaagtcgtgagaaaccatacatgcccaggtatggtcttcaacgcaacctgagagatcaaagtttggcaaggtatgcttttgatttctatgagatcactgcaaccactccgatcagagccaaagaggcgcatctgcagatgctagcagcagcgctgaagaattcgaacactaatatgtttggactggacggaaatgtcacaacttcagaagaggacacagaaaggcacacagcaacggatgtatcgcaacatgcatcaccttttaggcgtgaagggggtgt

SEQ ID NO: 20 - protein, 269 aa, Potato virus A
AETLDASEALAQKSEGRKKEEESNSSKAVAVKDKDVDLGTAGTHSVPRLKSMTSKLTLPMLKGKSVVNLDHLLSYKPKQVDLSNARATHEQFQNWYDGVMASYELEESSMEIILNGFMVWCIENGTSPDINGVWTMMDNEEQVSYPLKPMLDHAKPSLRQIMRHFSALAEAYIEMRSREKPYMPRYGLQRNLRDQSLARYAFDFYEITATTPIRAKEAHLQMLAAALKNSNTNMFGLDGNVTTSEEDTERHTATDVNRNMHHLLGVKGV

(19) United States
(12) Patent Application Publication
(10) Pub. No.: US 2013/0224235 A1
(43) Pub. Date: Aug. 29, 2013

(54) POTATO VIRUS A COAT PROTEIN-BASED VACCINES FOR MELANOMA

(71) Applicants: Lilian Järvekülg, Tallinn (EE); Viiu Paalme, Tallinn (EE); Ülo Puurand, Tallinn (EE); Sirje Rüütel Boudinot, Tallinn (EE); Sulev Kuuse, Tartu (EE); Reet Rumvolt, Vaana-Joesuu (EE); Ave Laas, Kohila vlad (EE)

(72) Inventors: Lilian Järvekülg, Tallinn (EE); Viiu Paalme, Tallinn (EE); Ülo Puurand, Tallinn (EE); Sirje Rüütel Boudinot, Tallinn (EE); Sulev Kuuse, Tartu (EE); Reet Rumvolt, Vaana-Joesuu (EE); Ave Laas, Kohila vlad (EE)

(73) Assignees: University of Tartu, Tartu (EE); Tallinn University of Technology, Tallinn (EE)

(21) Appl. No.: 13/124,741

(22) PCT Filed: Oct. 19, 2009
(86) PCT No.: PCT/EE2009/000016

§ 371 Date: Sep. 23, 2011

Related U.S. Application Data
(60) Provisional application No. 61/106,164, filed on Oct. 17, 2008.

(51) Int. Cl. A61K 39/12

(52) U.S. Cl. CPC A61K 39/12
USPC 424/186.1; 424/204.1; 424/205.1; 424/196.11

(57) ABSTRACT

A vaccine for treating melanoma, comprising at least one melanoma antigen and a viral-like particle comprising potato virus A coat protein. Use of vaccine for treating melanoma, including protein and DNA vaccines, based on potato virus A coat protein.

### FIELD OF THE INVENTION

The present invention relates to a viral particle bearing immunogens and having immunopotentiation activity. The invention particularly relates to bacterially expressed and purified chimeric virus-like particles (VLPs) as a vaccine and to a eukaryotic DNA vaccine based on suitable recombinant expression vector giving rise VLPs in vivo after recombinant DNA transfection of mammalian cells and method for enhancing an immune response in a human or an animal by means of these particles.

The present invention relates to the field of vaccine formulations and, in, particular, to melanoma vaccines based on plant virus (such as potyvirus) virus-like particles. The melanoma antigenic peptide (epitope) from gp100, a melanoma antigen can be genetically fused to a coat protein (CP) of the potato virus A (PVA) VLPs.

### BACKGROUND OF THE INVENTION

Rational design of vaccine delivery to the appropriate target cells is extremely important in the modern design of vaccines for the treatment of recalcitrant infections diseases and cancer. Among the numerous new approaches to vaccine development, VLPs made of viral nucleocapsids have emerged as a promising strategy.

To date, two VLP vaccines, hepatitis B virus (HBV) and Human Papilloma Virus (HPV), have been shown to function efficiently in humans (Fagan et al., 1987, Harper et al., 2004). VLPs made from the human papillomavirus (HPV) major capsid protein L1, for example, were shown to provide 100% protection in woman against development of cervical cancers (Ault, K. A., 2006; Harper et al., 2004; see also U.S. Pat. No. 7,279,306).

Platforms such as bacteriophage Qβ (Maurer et al., 2005), hepatitis B virus VLPs made of the viral core protein (Mihailova et al., 2006; Pumpens et al., 2002), and parvovirus VLPs (Antonis et al., 2006; Ogasawara et al., 2006) have also shown capacity to carry epitopes and to induce strong immune responses.

In recent years, the use of plant virus-derived VLPs as novel system for the expression of foreign epitopes and for the development of new vaccines has triggered much interest. Plant viruses strongly stimulate humoral and cellular immunity because of its structural and physical properties. In addition, they are phylogenetically distant from the animal immune system, which makes them good candidates for the development of vaccines. By genetically modifying these viruses, immunogenic peptides can be fused efficiently to the coat protein (CP) and exposed on the surfaces of the assembled plant VLPs. For example, cowpea mosaic virus (CPMV), tobacco mosaic virus (TMV), and alfalfa mosaic virus (AIMV) have been modified for the presentation of epitopes of interest (Canizares, et al., 2005; Brennan et al., 2001).

WO9739134 describes chimeric virus-like particles that comprise a coat protein and a non-viral protein, which can be used, for example, for presentation of peptide epitopes. WO0166778 describes a plant virus coat protein, and specifically a tobamovirus coat protein, fused via a linker at the N-terminus to a polypeptide of interest, which may include an epitope of a pathogenic micororganism. WO0200169 describes vaccines comprising either potato virus Y coat protein or a truncated bean yellow mosaic virus coat protein fused to a foreign peptide, and specifically a Newcastle Disease Virus or human immunodeficiency virus (HIV) epitope. Also, U.S. Pat. No. 6,448,070 describes methods of administering fusions of polypeptides, such as pathogen epitopes with alfalfa mosaic virus or ilarvirus capsid proteins, to an animals in order to raise an immune response.

VLPs derived from Potato Virus X (PVX) carrying an antigenic determinant from HIV (gp41 or gp120), HCV (NS3, E1 or E2), EBV (EBNA-3A, 3B or 3C) or the influenza virus (matrix protein or haemagglutinin) have been described (EP1167530). The ability of the PVX VLP carrying an HIV epitope to induce antibody production in mice via humoral and cell-mediated pathways. Additional adjuvants were used in conjunction with the PVX VLP to potentiate this effect.

However, there are known only few examples of genetically altered plant virus capsid proteins expressed in heterologous systems and used as foreign peptide carriers—coat proteins of Johnson grass mosaic virus (JGMV) (Jagadish et al., 1993; 1996, Saini and Vrati 2003) and Papaya ringspot virus (PRSV) (Chatchen et al., 2006) and very recently, VLPs derived from the coat protein of papaya mosaic virus (PapMV) and their use as immunopotentiators has been described (WO2004004761 and WO2008058396).

Experiments with JGMV potyvirus revealed that hybrid CPs containing either short peptides or short antigens fused at the N- or C-termini retained the ability to assemble into chimeric potyvirus-like particles. The chimeric potyvirus VLPs were highly immunogenic in mice and rabbits (Jagadish et al., 1996). Saini and Vrati (2003) described the fusion of Japanese encephalitis virus (JEV) major envelope protein E protein-derived peptides to the C-terminally truncated coat protein of the JGMV. Potyvirus-like particles of JGMV assembled after the expression of the fusion proteins in E. coli. The recombinant CP was shown to autoassemble and form VLPs. Immunization with chimeric VLPs containing a 27-mer peptide induced neutralizing antibodies in mice and protected the animals from lethal challenge by JEV in the absence of adjuvant.

Another potyvirus PRSV CP has proven to be effectively used for antigen presentation when produced in E. coli. 15-aa peptide epitope of the CPV VP2 protein was fused to the N- and C-termini of the PRSV CP, mice immunized with purified recombinant CPs elicited strong immune response against CPV (Chatchen et al., 2006).

These data demonstrate that plant virus-based VLPs can trigger a protective antiviral T-cell response. Thus, it has proven that peptides presented by these VLPs are highly immunogenic. Further improvements of the system may facilitate its use as a source of alternative vaccines, possibly as both prophylactic and therapeutic, and possibly as vaccines against chronic viral infections and cancer.

Conventional treatment options for malignant tumors include surgery, chemotherapy, and radiation. Because these treatment approaches are highly invasive and sometimes have only a palliative effect, alternative options to prevent or to treat malignant tumors are currently under investigation. In recent years, increasing efforts have been made to use vaccination strategies, including genetically modified tumor cells, dendritic cells either pulsed or transduced with tumor-associated antigens, immunization with soluble proteins or synthetic peptides, recombinant viruses or bacteria encoding tumor-associated antigens, and naked plasmid DNA encoding tumor-associated antigens (Haupt et al., 2002; Bocchia et al., 2000). All of these antitumor vaccination approaches aim to induce specific immunological responses to tumor-associated antigens, destroying tumor cells and protecting patients from relapses. A persistent antitumor immune memory is based on the induction of expanded populations of T or B lymphocytes, which first recognize and then react against tumor-associated antigens with specificity and high destructive potential (Ada G. L., 1990). One novel and promising strategy for antitumor vaccination is the direct inoculation of plasmid DNA encoding tumor-associated antigens. This technique, called DNA immunization, is known to induce both antigen-specific cellular as well as humoral immune responses (Weiner et al., 1999; Pardoll et al., 1995 and Donnelly et. al., 1997). The generation of T cell-mediated cytotoxicity or antibody-mediated cytotoxicity against tumor cells can inhibit tumor growth and lead to tumor rejection. Moreover, it is possible to combine DNA and protein vaccination and, in some cases, this approach has been shown to significantly increase the antitumor efficacy (Zöller et al., 2001). In contrast to peptide-based immunization approaches, which usually offer only a limited number of epitopes, DNA immunization allows the involvement of multiple different antigenic epitopes and a broad range of MHC restriction. Thus, DNA vaccination does not require prior knowledge of host haplotypes.

It is of interest to note that combining DNA and peptide vaccination strategies may have a synergistic effect in antitumor therapy (Nawrath et al., 2001).

### SUMMARY OF THE INVENTION

An object of the present invention is to provide potato virus A-based vaccines for melanoma. In accordance with one aspect of the invention, there is provided an antigen-presenting system (APC) comprising an epitope from gp100, a melanoma antigen in combination with a viral-like-particle (VLP) comprising potato virus A (PVA) coat protein (CP).

One aim of the present invention is to provide an immunogen-carrier complex having an immupotentiation property, consisting of a viral-like particle (VLP) carrying at least one immunogen in fusion with a protein or fragment thereof of said VLP, that may be used in the preparation of a composition for inducing an immune response against the protein or fragment thereof.

Another aim of the present invention is to provide a composition comprising a viral-like particle (VLP) and a protein or fragment thereof that may be used as a vaccine.

One embodiment of the present invention provides a protein vaccine—a PVA CP VLPs that can include an epitope from the melanoma antigen gp100. Another embodiment of the invention provides a DNA vaccine that can include the same melanoma epitope. It is possible to combine DNA and protein vaccination, and this approach increases the antitumor efficacy and may have a synergistic effect in antitumor therapy.

In accordance with the present invention there is also provided a method for immunopotentiating an immune response in a human or an animal which comprises administering to said human or animal an immunogen-carrier consisting of a viral-like particle (VLP) carrying at least one immunogen (tumor associated melanoma antigenic peptide (epitope) in fusion with a CP protein thereof of said VLP, independently or in combination with DNA vaccine (a naked DNA plasmid coding for PVA CP and fused to the antigenic melanoma peptide and delivered by gene gun) to elicit antigen-specific immune response based on chimeric PVA CP VLPs (bacterial or/and mammalian VLPs, respectively).

In accordance with the present invention there is provided a method of inducing an immune response against a melanoma, said method comprising administering to an animal an effective amount of a composition comprising one or more antigen-presenting systems (including DNA vaccine), in each of said antigen-presenting system comprising one or more melanoma epitopes.

In accordance with the present invention there is provided a use of an antigen presenting system(s) of the invention, in the manufacture of a medicament.

### BRIEF DESCRIPTION OF THE DRAWINGS

FIG. 1 presents the results of protein expression detection of transfected pcDNAs in mammalian cells. PK-15A cells transfected with recombinant pcDNAs. Cell nucleuses are stained with blue dye and expressed PVA CP fusion proteins are detected with anti-PVA antibodies and stained with red dye (shown with arrows). Panels: A and B-cells expressing wtCP/pcDNA construct; C and D-cells expressing

CP-gp-Nt/pcDNA construct (gp100 peptide fused to CP N-terminus), E and F-cells expressing CP-gp-Ct/pcDNA (gp100 peptide fused to CP C-terminus) construct.

Cells were examined at 100× magnification with “dapi” and “red fluorescence” filters.

FIG. 2 presents the results of expression of recombinant pQEs in E. coli. The cell lysates were separated on 12% SDS-PAGE gel, 6 μl sample load. Lanes: 1) PageRuler™ Prestained Protein Ladder (Fermentas), size shown in kDa; 2)-4) E. coli lysates of uninduced cells harboring wtCP/pQE construct, N-terminal construct CP-gp-Nt/pQE, C-terminal construct CP-gp-Ct/pQE, respectively; 5)-7) E. coli lysates of induced cells with wtCP/pQE construct, N-terminal CP-gp-Nt/pQE or C-terminal construct CP-gp-Ct/pQE respectively.

FIG. 3 illustrates the results of purification of recombinant proteins expressed in E. coli. 6 μl sample loads on 12% SDS-PAGE gel.

Purification under denaturing contitions: Panels A) wtCP protein; B) N-terminal fusion protein; C) C-terminal fusion protein. Lanes in all three panels: 1) PageRuler™ Prestained Protein Ladder; 2) E. coli lysate before applying to Ni-NTA purification column; 3)-6) Elution fractions 1-4 (buffer D) of the purified protein; 7)-9) Elution fractions 5-8 (buffer E) of the purified fusion proteins.

Purification under native conditions: Panels D) wt CP protein; E) N-terminal fusion protein; F) C-terminal fusion protein. Lanes in all three panels: 1) PageRuler™ Prestained Protein Ladder; 2) E. coli lysate before applying to Ni-NTA purification column; 3) E. coli lysate-Ni-NTA mixture column flow-through; 4) and 5) Column wash fractions 1-2 (wash buffer); 6)-9) Elution fractions 1-4 (elution buffer) of the purified protein.

FIG. 4 illustrates the results of protein purification under native and denaturing conditions after dialysis; 12% SDS-PAGE gel, load 5 μl. Lanes: 1) PageRuler™ Prestained Protein Ladder; 2) wtCP protein purified under native conditions, 3) and 4) wtCP protein purified under denaturing conditions, merged elution fractions E2-E5 and E6-E8, respectively; 5) N-terminal fusion protein purified under native conditions; 6) and 7) N-terminal fusion protein purified under denaturing conditions, merged elution fractions E2-E5 and E6-E8, respectively; 8) C-terminal fusion protein purified under native conditions; 9) and 10) C-terminal fusion protein purified under denaturing conditions, merged elution fractions E2-E5 and E6-E8, respectively.

FIG. 5 presents filamentous PVA CP VLPs produced in E. coli cells, purified under native conditions; Preparates were examined at 30000× magnification under transmission electron microscope. Panels—A: VLPs formed by wtCP construct; B: VLPs formed by N-terminal fusion protein; C: VLPs formed by C-terminal fusion protein.

FIG. 6 illustrates Western blot analysis of recombinant proteins purified under native conditions and dialysed. Lanes: 1) PageRuler™ Prestained Protein Ladder; 2) wtCP; 3) N-terminal fusion protein; 4) C-terminal fusion protein.

FIG. 7 illustrates mean differences in responses against KLH-peptide and pure KLH (Keyhole limpet hemocyanin). For all serum groups, bars indicate the medium differences (response against KLH-peptide—response against KLH), optical density is shown on vertical axis, measured at wavelength 450 nm. Minimal and maximal difference values are shown by vertical lines on the bars. WtCP: results for all serums raised against wtCP protein, the medium of 7 serum tested; N-t: results for all serums raised against N-terminal protein, the medium of 9 serums tested; C-t: results for all serums raised against C-terminal protein, the medium of 3 serums tested; Control: results for all serums raised against PBS, the medium of 4 serums tested.

FIG. 8 presents the titration curves of the serums tested. One serum of each group is shown in this figure. All serums were tested against pure. KLH and KLH-peptide, both curves are shown. wt-serum is the serum raised against wtCP protein, N-serum is the serum raised against N-terminal protein, C-serum is the serum raised against the C-terminal protein, Contr is the control serum, raised against PBS. Serums against CP and CP fusion proteins are compared to control serum.

Based on these titration curves (FIG. 8), the dilution 1:100 was chosen for presenting results from indirect ELISA.

FIG. 9a presents the results. IL-4 and FIG. 9b INF-γ producing lymphocytes were detected after stimulation with synthetic gp100_{(209-217(2M)} peptide IMDQVPFSV, the same sequence presented on the surface of recombinant CP fusion protein. N1, N2, N3 and N4-spleen cells from mice immunized with N-terminal fusion protein; WT-spleen cells from mice immunized with wild type CP (wtCP) protein; PBS-spleen cells from control mice immunized with PBS. The mean cell counting results are shown in this figure, 4×10⁶ cells were added to each well, cells from one mouse immunized with wtCP protein, cells from four mice immunized with N-terminal fusion protein and cells from one control mouse immunized with PBS are involved in this experiment, all in two parallels.

FIG. 10 presents the results of survival rates in all test groups shown in percentages. Days after melanoma challenge are shown on the vertical axes; ten mice were in control group and wtCP group, nine mice in N-terminal fusion protein group.

FIG. 11 illustrates the relative sizes of melanomas developing in all three test groups. The sizes of melanomas were measured in millimeters, the average size of melanomas in entire groups (negative mice included in calculation) is shown in this figure. Mice who had died or were euthanized, are included and the size of melanoma on the last measurement was taken into account. Days after melanoma challenge are shown on the vertical axes; ten mice were in control group and wtCP group, nine mice in N-terminal protein group.

FIG. 12 presents the liquid RNA binding assay of his-tagged CP point mutants. Different amounts of purified proteins were mixed with 10⁶ cpm of 32P labeled RNA sense transcripts (corresponding to the CP gene of PVA RNA) in RNA binding buffer in the presence of 100 mM NaCl, incubated 30 mM at RT, and filtered through GF/C filters. The washed filters were counted for β-radiation. The bars represent the mean±standard error of three experiments.

FIG. 13 presents the detection of amplification of different PVA-B11 CP mutants in tobacco protoplasts 48 h postelectroporation with 35S DNA constructs. Viral negative strand RNA from total RNA extracts was detected by RNA dot blot hybridization using virus specific RNA probes complementary to the viral CP region. Equal amounts of RNA were dotted on the membrane. Lane 1, mock-inoculated protoplasts; 2, wt PVA-B11; 3-6, the four CP mutants.

### DETAILED DESCRIPTION OF THE INVENTION

An antigen-presenting system (APS) comprising one or more melanoma antigenic peptides (epitopes) from gp100, a melanoma antigen in combination with a VLP derived from potato virus A coat protein subunits is provided.

An antigen-presenting system (APS) comprising procaryotic and/or eukaryotic expression vectors for VLP expression is provided.

In accordance with one embodiment of the present invention, the APS is capable of inducing a humoral immune response, a cellular immune response, or both, in an animal. The APS is thus suitable for use as a vaccine, which may require an active participation of one or both of these two branches of the immune system.

In one embodiment of the present invention, the APS comprises one or more melanoma epitopes and is suitable for use as a vaccine for melanoma.

### DEFINITIONS

Unless defined otherwise, all technical and scientific terms used herein have the same meaning as commonly understood by one of ordinary skill in the art of this invention.

The ten “adjuvant” as used herein, refers to an agent that augments, stimulates, potentiates and/or modulates an immune response in an animal. An adjuvant may or may not have an effect on the immune response in itself.

As used herein, a “chimeric protein” is a protein that is created when two or more genes that normally code for two separate proteins or protein fragments recombine, either naturally or as the result of human intervention, to provide a polynucleotide encoding a protein (the “chimeric protein”) that is a combination of all or part of each of those two proteins. In the context of the present invention, a “fusion protein” is considered to be a “chimeric protein”.

The expression “fusion coat protein” is used herein to refer to a fusion protein in which one of the proteins in the fusion is a Potato Virus A (PVA) coat protein.

The terms “immunogen”, “antigen” and “antigenic peptide (epitope)” as used herein refer to a portion or portions of molecules which are capable of inducing a specific immune response in a subject alone or in combination with an adjuvant.

The term “immune response”, as used herein, refers to an alteration in the reactivity of the immune system of an animal in response to an antigen or antigenic material and may involve antibody production, induction of cell-mediated immunity, complement activation, development of immunological tolerance, or a combination thereof.

The term “immunoprotection” as used herein, mean an immune response that is directed against one or more antigen so as to protect against disease and/or infection by a pathogen in a vaccinated animal. For purposes of the present invention, protection against disease includes not only the absolute prevention of the disease, but also any detectable reduction in the degree or rate of disease, or any detectable reduction in the severity of the disease or any symptom in the vaccinated animal as compared to an unvaccinated infected or diseased animal. Immunoprotection can be the result of one or more mechanisms, including humoral and/or cellular immunity.

The terms “polypeptide” or “peptide” as used herein is intended to mean a molecule in which there is at least four amino acids linked by peptide bonds.

The expression “viral nucleic acid”, as used herein, may be the genome (or a majority thereof) of a virus, or a nucleic acid molecule complementary to viral RNA is also considered viral nucleic acid, as is a RNA molecule that is complementary in base sequence to viral DNA.

The term “virus-like particle” (VLP), as used herein, refers to a self-assembling particle which has a similar physical appearance to a virus particle. The VLP may or may not comprise viral nucleic acids. VLPs are generally incapable of replication.

The term “pseudovirus” or “mammalian VLPs”, as used herein, refers to a VLP that comprises nucleic acid sequences, such as DNA or RNA, including nucleic acids in plasmid form. Pseudoviruses are generally incapable of replication.

The term “vaccine”, as used herein, refers to a material capable of producing an immune response.

### Antigen-Presenting System (APS)

An antigen-presenting system (APS) of the present invention comprises one or more epitopes from melanoma gp100 antigen in combination with a virus-like particle (VLP) derived from a Potato virus A (PVA) coat protein. By “derived from” it is meant that the VLP comprises coat proteins that have an amino acid sequence substantially identical to the sequence of the wild-type coat protein and may optionally include one or more antigens attached to the coat protein, as described in more detail below. The PVA coat protein can comprise the sequence of the wild-type coat protein or a modified version thereof which is capable of multimerization and self-assembly to form a VLP. In one embodiment of the present invention the APS comprises one or more peptides from the melanoma antigens.

The one or more epitopes comprised by the APS can be conjugated to a coat protein of the PVA VLP. Conjugation can be, for example, by genetic fusion with the coat protein.

The PVA VLP included in the APS thus acts as an immunopotentiator capable of potentiating an immune response in an animal, and optionally as a carrier for the antigen(s) comprised by the APS. In accordance with one embodiment of the present invention, the APS is capable of inducing a humoral and/or cellular immune response in an animal and thus is suitable for use as vaccines, for example a melanoma vaccine.

### Potato Virus A (PVA) and PVA CP VLPs

Potato virus A (PVA) is a filamentous RNA virus which belongs to the genus Potyvirus (family Potyviridae), the largest group of plant viruses. Potyvirus particles are flexible rods, 680-900 nm long and 11.15 nm wide, consisting of more than 2000 copies of a coat protein subunits and one single-stranded RNA genome. The potyvirus coat protein (CP) is a multifunctional protein which plays a role in virus transmission by aphids, virus movement in plants and virion formation. Three regions can be distinguished in the potyviral CP protein. The central core region involves 214-217 amino acids (aa) and is considered to be responsible for particle integrity. Both the N-terminal and C-terminal regions are surface exposed and are not required for virion assembly. The expression of potyvirus CP in bacteria (E. coli), yeast (S. cerevisiae), insect cells or mammalian cell culture has been demonstrated to result in the formation of potyvirus-like particles (PVLPs). It has been shown that self-assembly of the particles is not impaired, even by the insertion of a foreign 26 kD protein into the Johnsongrass mosaic potyvirus CP (Jagadish et al 1996). Such chimeric PVLPs have been highly immunogenic, even in the absence of any adjuvant.

Recently, it has been shown the potential of fusion genes linking the sequence coding for the human papillomavirus 16 E7 (HPV16 E7) immunodominant epitope to the PVA CP gene to induce E7-specific cellular immune response in the mouse model (Pokorna et al., 2005).

The APS of the present invention comprises PVA CP VLPs which are formed from recombinant PVA coat proteins that have multimerised and self-assembled to form a VLP. When assembled, each VLP comprises a long helical array of coat protein subunits. The wild-type virus comprises over 2000 coat protein subunits and is about 800 nm in length. PVA VLPs that are either shorter or longer than the wild-type virus can still, however, be effective.

### Melanoma Antigens

In accordance with one embodiment of the present invention, the APS comprises one or more melanoma antigenes. The antigen may be an immunogen, epitope, mimotope or antigenic determinant derived from the melanoma antigens and may be, for example, a peptide, a protein, a nucleic acid, or combination thereof derived from the melanoma antigens.

Certain human cancers have been found to express tumor-associated antigens (TAAs) that can be recognized immunologically. The antigens identified so far from human melanoma (herein call melanoma antigens) can be divided into two classes based on their expression pattern. The antigens of the first class are encoded by genes that are expressed only in tumor and testis, but not other normal human tissues. MAGE1, MAGE3, GAGE and BAGE are examples of this class.

The second class of antigens represents differentiation antigens encoded by genes that are expressed only in melanocytes, melanomas, and normal retina. MART-1/Melan-A, gp100 and tyrosine are examples of this class. All these antigens are nonmutated self proteins. However, several mutated antigens were also identified to be recognized by T cells, including CDK4, β-catenin and Mum-1 (WO2008058396). Identification of the antigenic epitopes recognized by T cells derived from the corresponding gene products is important for developing new, effective immunotherapeutic strategies with these antigens or synthetic peptides for the treatment of patient with cancer.

### Melanoma Antigen Gp100

Tumor differentation antigen gp100 is a product of the Silver locus, it is a glycoprotein that is in humans expressed both by normal melanocytes and the majority of malignant melanomas tested with PMEL-17 as mouse homologue. Gp100 is a 661-amino acid melanosomal matrix protein involved in melanin synthesis that was cloned during the attempt to isolate tyrosinase encoding gene. It was initially identified as a potential tumour rejection antigen recognized by T-cells because of its recognition by tumour-infiltrating lymphocytes, that are associated with cancer regression in melanoma patients (Bakker et al., 1994).

Ten different gp100 peptide epitopes have been described to be presented on HLA-A*0201, although only three of these peptides gp100₂₀₉₋₂₁₇, gp100₂₈₀₋₂₈₈ and gp100₁₅₄₋₁₆₂ were recognized by tumour-infiltrating lymphocytes. Recognition of the gp100₂₀₉₋₂₁₇ epitope by adoptively transferred tumour-infiltrating lymphocytes has been associated with tumour regression. A modified version of this gp100₂₀₉₋₂₁₇ epitope IMDQVPFSV, gp100_{209-217(2M)}, in which threonine at second position has been replaced with methionine, is one of the first modified melanoma peptides tested in clinical trials, being more immunogenic in melanoma patients and in mice (Rosenberg et al., 1998a), most probably due to considerably lower rate of dissociation from the HLA-A*0201 molecule compared to the native peptide, resulting in increased stability of the peptide-MHC complex necessary for immune stimulation (Yu et al., 2004). The affinity of the modified gp100_{209-217(2M)} peptide for HLA-A*0201 has been reported to be approximately 9 times stronger than that of the parental peptide (Engelhard et al., 2002). Using gp100_{209-217(2M)} peptide for vaccination of melanoma patients has proven to induce successful immunization in a majority of the patients (91% in Rosenberg et al., 1998a; 94% in Walker et al., 2004). Moreover, 13 of 31 patients (42%) had objective cancer responses and the maintenance of long-term memory cells was observed that were capable of accelerated proliferation response and were functionally active after in vitro stimulation expansion (Walker et al., 2004). These data demonstrate this modified self-peptide to be a powerful means of immunizing patients and this response can include the induction of long-lived memory T-cells. However, using full-length gp100 glycoprotein as vaccine, the substitution of a T to a M at position 210 does not significantly alter the efficiency of antigen presentation (Nagorsen et al., 2004).

In present invention a modified version of gp100₂₀₉₋₂₁₇ epitope IMDQVPFSV (Rosenberg et al., 1998a) was tested and herein we designated it as melanoma antigenic peptide (epitope) from gp100, a melanoma antigen.

In one embodiment of the present invention, the epitope is a peptide derived from the gp100, a melanoma antigen. The epitope may be specific or recognized by surface structures on T cells, B cells, NK cells and macrophages, or Class I or Class II APC associated cell surface structures. In one embodiment, the invention is especially useful for small weakly immunogenic antigens.

As noted above, the antigens comprised by the APS can be entire proteins or fragments thereof. Thus, the APS of the present invention may comprise one or more antigens each having a single epitope capable of triggering a specific immune response.

In accordance one embodiment of the present invention, the one or more epitopes are conjugated to a coat protein of a PVA VLP. As the VLP comprises multiple copies of self-assembled coat protein, attaching the epitopes to the coat protein allows presentation of the epitopes in an organized fashion on the surface of the VLP. In accordance with this embodiment, the VLP comprises coat proteins that include a first portion that is a recombinant PVA coat protein conjugated to a second portion that comprises one or more epitopes. The antigen-conjugated coat protein retains the ability to assemble with other fusion coat proteins or with wild-type coat protein to form an immunogen-carrier VLP.

In order to allow presentation of the epitope on the surface of the VLP and enhance immune recognition of the epitope, the antigen is preferably attached to a region of the coat protein that is disposed on the outer surface of the VLP. Thus the antigen can be inserted near, or attached at, the amino- (N-) or carboxy- (C-) terminus of the coat protein, or it can be inserted into, or attached to, an internal loop of the coat protein which is disposed on the outer surface of the VLP. In one embodiment of the present invention, the epitope is present at, or proximal to, the N-terminus of the PVA coat protein.

In accordance with one embodiment of the invention, the APS comprises a PVA coat protein genetically fused to one or more epitopes. The epitope can be directly fused to the coat protein sequence such that the sequences are contiguous, or a “spacer” of one or more amino acids can be inserted between the coat protein sequence and the sequence of the epitope.

### Preparation of the APS

The present invention provides APSs that comprise PVA VLPs derived from a recombinant PVA coat protein. The invention further provides recombinant PVAs VLPs that comprise one or more melanoma antigenic peptides (epitopes) from gp100, a melanoma antigen in genetic fusion with the coat proteins. These recombinant coat proteins are capable of multimerisation and assembly into VLPs. Methods of genetically fusing the epitopes to the coat protein are known in the art and some are described below and in the Example II.

### PVA CP VLPs

The present invention particularly relates to bacterially expressed and purified chimeric virus-like particles (VLPs) as a vaccine. The invention also relates to a eukaryotic DNA vaccine based on suitable recombinant expression vector giving rise VLPs in vivo after DNA transfection of mammalian cells.

The recombinant coat proteins for use to prepare the VLPs of the present invention can be readily prepared by standard genetic engineering techniques by the skilled worker provided with the sequence of the wild-type protein. Methods of genetically engineering proteins are well known in the art (Current Protocols in Molecular Biology, John Wiley & Sons, New York), as is the sequence of the wild-type PVA coat protein (see SEQ ID NO 1 and SEQ ID NO 2).

Isolation and cloning of the nucleic acid sequence encoding the wild-type protein can be achieved using standard techniques (Sambrook and Rusell, 2001). For example, the nucleic acid sequence can be obtained directly from the PVA by extracting RNA by standard techniques and then synthesizing cDNA from the RNA template (for example, by RT-PCR).

The nucleic acid sequence encoding the coat protein is then inserted directly or after one or more subcloning steps into a suitable expression vector. One skilled in the art will appreciate that the precise vector used is not critical to the instant invention. Examples of suitable vectors include, but are not limited to, plasmids, phagemids, cosmids, bacteriophage, baculoviruses, retroviruses or DNA viruses. The coat protein can then be expressed and purified as described in more detail in Example II.

Alternatively, the nucleic acid sequence encoding the coat protein can be further engineered to introduce one or more mutations, such as those described above, by standard in vitro site-directed mutagenesis techniques well-known in the art. Mutations can be introduced by deletion, insertion, substitution, inversion, or a combination thereof, of one or more of the appropriate nucleotides making up the coding sequence. This can be achieved, for example, by PCR based techniques for which primers are designed that incorporate one or more nucleotide mismatches, insertions or deletions. The presence of the mutation can be verified by a number of standard techniques, for example by restriction analysis or by DNA sequencing.

As noted above, the coat proteins can also be engineered to produce fusion proteins comprising one or more epitopes fused to the coat protein. Methods for making fusion proteins are well known to those skilled in the art. DNA sequences encoding a fusion protein can be inserted into a suitable expression vector as noted above.

One of ordinary skill in the art will appreciate that the DNA encoding the coat protein or fusion protein can be altered in various ways without affecting the activity of the encoded protein. For example, variations in DNA sequence may be used to optimize for codon preference in a host cell used to express the protein, or may contain other sequence changes that facilitate expression.

In the context of the present invention, the expression vector may additionally contain heterologous nucleic acid sequences that facilitate the purification of the expressed protein. Examples of such heterologous nucleic acid sequences include, but are not limited to, affinity tags such as metal-affinity tags, histidine tags, avidin/streptavidin encoding sequences, glutathione-S-transferase (GST) encoding sequences and biotin encoding sequences. The amino acids corresponding to expression of the nucleic acids can be removed from the expressed coat protein prior to use according to methods known in the art. Alternatively, the amino acids corresponding to expression of heterologous nucleic acid sequences can be retained on the coat protein if they do not interfere with its subsequent assembly into VLPs.

In one embodiment of the present invention, the coat protein is expressed as a histidine tagged protein. The histidine tag can be located at the carboxyl terminus or the amino terminus of the coat protein.

The expression vector can be introduced into a suitable host cell or tissue by one of a variety of methods known in the art. Such methods can be found generally described in Sambrook and Rusell 2001 and include, for example, stable or transient transfection, lipofection, electroporation, and infection with recombinant viral vectors. One skilled in the art will understand that selection of the appropriate host cell for expression of the coat protein will be dependent upon the vector chosen. Examples of host cells include, but are not limited to, bacterial, yeast, insect, plant and mammalian cells. The precise host cell used is not critical to the invention. The coat proteins can be produced in a prokaryotic host (e.g., E. coli, A. salmonicida or B. subtilis) or in a eukaryotic host (e.g., Saccharomyces or Pichia; mammalian cells, e.g., porcine kidney cells (PK-15), COS, NIH 3T3, CHO, BHK, 293, or HeLa cells; or insect cells). In one embodiment, the coat proteins are expressed in prokaryotic cells and/or in eukaryotic cells.

If desired, the coat proteins can be purified from the host cells by standard techniques known in the art (see, for example, in Current Protocols in Protein Science, ed. Coligan, J. E., et al, Wiley & Sons, New York, N.Y.) and sequenced by standard peptide sequencing techniques using either the intact protein or proteolytic fragments thereof to confirm the identity of the protein.

The recombinant coat proteins of the present invention are capable of multimerisation and assembly into VLPs. In general, assembly takes place in the host cell expressing the coat protein. The VLPs can be isolated from the host cells by standard techniques, such as those described in the Examples section provided herein. In general, the isolate obtained from the host cells contains a mixture of VLPs, discs, less organised forms of the coat protein (for example, monomers and dimers). The VLPs can be separated from the other coat protein components by, for example, ultracentrifugation to provide a substantially pure VLP preparation. In this context, by “substantially pure” it is meant that the preparation contains 70% or greater of VLPs. Alternatively, a mixture of the various forms of coat protein can be used in the final vaccine compositions. When such a mixture is employed, the VLP content should be 40% or greater.

The VLPs can be further purified by standard techniques, such as Ni-NTA affinity chromatography, to remove contaminating host cell proteins or other compounds, such as LPS. In one embodiment of the present invention, the VLPs are purified to remove LPS.

### Characteristics of Recombinant Coat Proteins

The recombinant coat proteins can be analysed for their ability to multimerize and self-assemble into a VLP by standard techniques. For example, by visualising the purified recombinant protein by electronmicroscopy (see, for example, Example II).

Stability and purity of the VLPs can be determined if desired by techniques known in the art, for example, by SDS-PAGE analyses (see Example II).

### Evaluation of Efficacy

In order to evaluate the efficacy of the APSs of the present invention as vaccines, challenge studies can be conducted. Such studies involve the inoculation of groups of test animals (such as mice) with an APS of the present invention by standard techniques. Control groups comprising non-inoculated animals and/or animals inoculated with a commercially available vaccine, or other positive control, are set up in parallel. After an appropriate period of time post-vaccination, the animals are challenged with a melanoma cells. Blood samples collected from the animals pre- and post-inoculation, as well as post-challenge are then analyzed for an antibody response and/or T cell response to the melanoma antigens. Suitable tests for the B and T cell responses include, but are not limited to, Western blot analysis and Enzyme-Linked Immunosorbent Assay (ELISA) assay. Cellular immune response can also be assessed by techniques known in the art, including monitoring T cell expansion and IFN-γ secretion release, for example, by ELISPOT to monitor induction of cytokines (see Example II).

The animals can also be monitored for development of other conditions associated with infection with melanoma including, for example, growing melanoma size, and the like for certain melanoma lines, survival is also a suitable marker.

### Vaccine Compositions

The present invention provides for compositions suitable for use as melanoma vaccines comprising one or more APS of the invention together with one or more non-toxic pharmaceutically acceptable carriers, diluents and/or excipients. If desired, other active ingredients, adjuvants and/or immunopotentiators may be included in the compositions.

The compositions can be formulated for administration by a variety of routes. For example, the compositions can be formulated for parenteal, topical, or rectal administration. The term parenteral as used herein includes subcutaneous injections, intravenous, intramuscular, intrathecal, intrasternal injection or infusion techniques.

In one embodiment of the present invention, the compositions are formulated for topical administration using gene gun. In another embodiment, the compositions are formulated for parenteral administration.

The compositions preferably comprise an effective amount of one or more APS of the invention. The term “effective amount” as used herein refers to an amount of the APS required to induce a detectable immune response. The effective amount of APS for a given indication can be estimated initially, for example, either in cell culture assays or in animal models, usually in rodents, rabbits, dogs, pigs or primates. The animal model may also be used to determine the appropriate concentration range and route of administration. Such information can then be used to determine useful doses and routes for administration in the animal to be treated, including humans. In one embodiment of the present invention, the unit dose comprises between about 25 μg to about 50 μg of protein. In another embodiment, the unit dose comprises between about 0.5 to about 1 μg of cDNA. One or more doses may be used to immunise the animal, and these may be administered on the same day or over the course of several days or weeks. In one embodiment of the invention, two or more doses of the composition are administered to the animal to be treated. In another embodiment, three or more doses of the composition are administered to the animal to be treated.

One embodiment of the present invention provides a protein vaccine—a PVA CP VLPs that can include an antigen from the melanoma antigen gp 100 and/or NY-ESO-1. Another embodiment of the invention provides a DNA vaccine that can include the same melanoma antigens. It is possible to combine DNA and protein vaccination, and this approach increases the antitumor efficacy and may have a synergistic effect in antitumor therapy.

### Applications and Uses

The present invention provides for a number of applications and uses of the APSs described herein. Non-limiting examples include the use of the APS as a vaccine against melanoma and/or to potentiate an immune response against a melanoma antigen(s), and the use of the APS to screen for antibodies to melanoma. The present invention thus also provides methods for potentiating and/or inducing an immune response to melanoma antigen(s) in an animal. As well, the use of PVA CP VLPs and APSs of the invention for the preparation of medicaments, including vaccines, and/or pharmaceutical compositions is within the scope of the present invention. As demonstrated herein, PVA CP VLPs are capable of potentiating both a humoral and a CTL response to an antigen.

The present invention also provides for the use of the APS as a screening agent, for example, to screen for antibodies to melanoma cells. The APS can be readily adapted to conventional immunological techniques such as an enzyme-linked immunosorbant assay (ELISA) or Western blotting and is thus useful in diagnostic and research contexts.

### EXAMPLES

### Example I

### Effect of Mutations on Self-Assembly of PVA Coat Protein and/or RNA Binding

### Materials and Methods

### Construction of Point Mutants of PVA CP.

Single aa substitution mutations were introduced into the coding sequence of the core region of the CP by site directed mutagenesis (Kunkel et al., 2001). A two step PCR based mutagenesis was performed in recombinant pUC57 subclones, the primers used in the mutagenesis were as follows:

| | |
|---|---|
| P1: | 5'-GGGTATGGTCTTCAACGCAACC-3' |
| P2: | 5'-GGGCATGTATGGTTTCTCACGAC-3' |
| P3: | 5'-GGAGCCAAAGAGGCGCATCTGC-3' |
| P4: | 5'-GATCGGAGTGGTTGCAGTGATCTC-3' |
| P5: | 5'-CTGATGAAAGCAGCAGCGCTGAAG-3' |
| P6: | 5'-CAGATGCGCCTCTTTGGCTCTGATCGG-3' |
| P7: | 5'-CTAGCAGCAGCGCTGAAGAATTCG-3' |
| P8: | 5'-CATCTGCAGATGCGCCTCTTTGGC-3' |

P1 and P2 were used to mutate Arg 185 to Gly, P3 and P4 to mutate Arg 214 to Gly, P5 and P6 to mutate Gln 221 to Leu and P7 and P8 to mutate Lys 223 to Leu. In all cases the universal forward and reverse primers were used as the outside primers in the PCR reactions. The presence of all the introduced mutations was confirmed by DNA sequence analysis

Transmission Electron Microscopy of VLPs Produced in E. coli.

Four CP point mutants expressed in E. coli were each (10 ul sample from cell lysate) placed on the carbon coated grids and stained with 2% uranyl acetate. Grids were air dried and examined at 35.000 magnification with electron microscope.

### Results

The full-length cDNA clone of potatovirus A isolate B11 driven by 35S promoter (pPVA-B11) has been described in (Puurand et al; 1996), which further was engineered with GFPuv reporter gene (Rajamäki et al., 1999 and 2005).

Point Mutations

in the CP gene were introduced into a full-length clone of pPVA-B11 in order to determine their effect on self-assembly of PVA CP and also to virus infectivity—genome replication, virion formation, and virus movement (cell-to cell and long distance). Single aa substitution mutations were introduced into the core region of the CP gene by site directed mutagenesis using mutagenic oligonucleotides. The following mutations were made: R185G, R214G, Q221L, K223L.

The mutagenized sequences were transferred a) into the full-length cDNA clone of PVA-B11, tagged with GFP reporter gene (Rajamäki et al 2005), to produce viral single mutants and b) the mutagenized CP genes were cloned in procaryotic expression vector pQE30 for recombinant CP expression and purification. Mutated coat proteins along with wtCP were expressed as 6×His-tagged recombinant proteins in Escherichia coli and purified to homogeneity under native conditions.

1. Liquid RNA-binding assay of his tagged CP point mutants was performed (FIG. 12) The results showed that mutations did not have any influence to RNA binding in vitro. CP mutants did bind RNA in vitro to the same degree as the wt protein.

2. The role of aforementioned four amino acids in protein-protein interactions was investigated with transmission electronmicroscopy. The recombinant proteins produced in E. coli autoassembled to form VLPs as shown by electron micrographs of cell lysates. The results from these experiments suggested that these four residues are not essential for the capsid assembly process.

3. PVA B11 full-length genome cDNA mutants were generated containing coat proteins with single amino acid substitutions (R185G, R214G, Q221L and K223L) to investigate their influence on the virus infectivity. ELISA was used to determine virus accumulation (e.g. long distance movement) in inoculated test plants. Based on the results of ELISA assay of the systemic leaves the four viral single mutants were not able to cause systemic infection in tobacco test plants.

4. RNA replication assay performed in tobacco protoplasts using coat protein mutants detected the negative strand RNA from total RNA extracts. Mutations did not have any negative effect on the amplification of the virus genome (FIG. 13).

5. Immunocapture-RT-PCR (IC-RT-PCR) proved that virus particles are formed in vivo in tobacco protoplasts, after electroporation of protoplasts with pPVA-B11 constructs, both with wt and mutated sequences.

Despite of normal RNA amplification activity of each CP mutant in protoplasts, they were impaired in establishing systemic infection in test plants. These results with viral mutants could be ascribed to defects in cell-to cell or long distance movement.

### Example II

### Development and Characterization of PVA Coat Protein Virus-Like Particles Carrying Melanoma Gp100 Peptide Aa209-217(2M)

### Materials and Methods

If not mentioned otherwise, standard methods (Sambrook and Russell, 2001) and protocols following the purchaser's recommendations were used.

### Virus Isolate, Bacterial Strain, Expression Vectors

The infectious cDNA clone pPVA-B11 (Puurand et al., 1996) was used as a template for polymerase chain reaction (PCR).

For maintenance and propagation of recombinant expression plasmids the Escherichia coli strain TG-1 was used, as well for prokaryotic expression of recombinant proteins. TG-1 strain contains lacI^{q} mutation and it produces enough lac repressor to block transcription. TG-1 strain is also suitable for storing and propagating both pQE and pcDNA plasmids and for expression of nontoxic proteins from pQE plasmids. Protein expression is regulated less tightly than in strains harboring pREP4 plasmid.

Bacterial cultures were cultivated in Luria-Bertani (LB) medium (10 mg/ml tryptone, 5 mg/ml yeast extract, 5 mg/ml NaCl, 1 mM NaOH), cultures containing recombinant plasmids in medium complemented with 100 μg/ml ampicillin, at temperature 37° C.

Vector used for production of recombinant proteins in prokaryotic systems was pQE-30 plasmid (Qiagen). The pQE-30 expression vectors provides high-level expression of proteins in E. coli, proteins are fused to 6×His affinity tag at the protein N-terminal end, that allows to immobilize the proteins on metal-chelating surfaces such as Ni-NTA Agarose (Ni-NTA coupled to Sepharose CL-6B, Qiagen). The 6×His tag is poorly immunogenic and at pH8.0 uncharged, affecting minimally the secretion and compartmentalization of folding of the protein. The recombinant protein can be used as antigen without removing the tag. pQE contains an optimized regulatable promotor-operator element consisting of T5 promotor and two lac operator elements, expression can be induced by addition of isopropyl-β-thiogalactopyranoside (IPTG) at final concentration 1 mM. pQE contains multi-cloning-site and translation stop-codons in all reading frames for convenient preparation of expression constructs.

Vector for producing recombinant proteins in eukaryotic systems was pcDNA3.0 (Invitrogen). pcDNA vectors are designed for high-level, constitutive protein expression in a variety of mammalian cell lines. Vector contains Cytomegalovirus (CMV) enhancer-promoter for high-level expression, T7, Sp6 and SV40 promoters, ampicillin resistance gene and pUC origin for selection and maintenance in E. coli, Simian vacuolating virus 40 (SV40) origin for episomal replication, Bovine Growth Hormone (BGH) polyadenylation signal and transcription terminator sequence for enhanced mRNA stability.

Preparation of Competent E. coli Cells

1.5 ml overnight (O/N) TG-1 E. coli culture was added to 50 ml SOB medium (20 mg/ml typtone, 5 mg/ml yeast extract, 10 mM NaCl, 2.5 mM KCl) complemented with 25 μl 2M MgCl₂ and 25 μl 2M MgSO₄, grown at 37° C. until OD₆₀₀=0.5. Culture was cooled on ice, centrifuged (Biofuge PrimoR, Heraeus) 10 minutes 3000 rpm at 4° C., supernatant was discarded. The pellet was resuspended in 1/3 of the initial culture volume of cold sterile RF1 (100 mM RbCl, 50 mM MnCl₂.4H₂O, 30 mM potassium-acetate, 10 mM CaCl₂.2H₂O, 150 mg/ml glycerol, pH 5.8) and incubated on ice for 15 minutes, centrifuged 10 minutes 3000 rpm at 4° C. Supernatant was discarded and pellet resuspended in 1/12 of the culture volume cold sterile RF2 (10 mM MOPS, 10 mM RbCl, 75 mM CaCl₂.2H₂O, 150 mg/ml glycerol, pH 6.8) and incubated on ice for 15 minutes. Competent cells were divided to aliquots, frozen in liquid nitrogen and stored at −70° C.

### Preparation of Expression Constructs Using Recombinant Primers

Table 1 presents the oligonucleotide primers used for PCR amplification. The restriction endonuclease sites are underlined, the 27 nucleotides coding for the melanoma gp100 epitope gp100_{209-217(2M)} (IMDQVPFSV) is, are presented in italic.

TABLE 1
Recombinant primers for preparation of expression constructs

| Primer name | Nucleotide sequence |
|---|---|
| CP-F for pQE | CACA GGATCC GCC GGA ACT CTT GAT GCA AGC G |
| CP-R for pQE | CACA GTCGAC TTA CAC CCC CTT CAC GCC TAG AAG G |
| CP-F for pcDNA | CACA GGATCC ATG GCC GGA ACT CTT GAT GCA AGC G |
| CP-R for pcDNA | CACA CTCGAG TTA CAC CCC CTT CAC GCC TAG AAG G |
| CP-F-gp for pQE | CACA GGATCC ATC ATG GAC CAG GTG CCG TTC TCC GTC ACA |
| | CCA GCG CAG AAA TCC GAA G |
| CP-F-gp for pcDNA | CACA GGATCC ATG ATC ATG GAC CAG GTG CCG TTC TCC GTC |
| | ACA CCA GCG CAG AAA TCC GAA G |
| CP-R-gp for pQE | CACA GTCGAC TTA GAC GGA GAA CGG CAC CTG GTC CAT GAT |
| | CAC CCC CTT CAC GCC TAG AAG G |
| CP-R-gp for pcDNA | CACA CTCGAG TTA GAC GGA GAA CGG CAC CTG GTC CAT GAT |
| | CAC CCC CTT CAC GCC TAG AAG G |

All together six different constructs of PVA CP and CP fused to melanoma gp100_{209-217(2M)} antigenic peptide were prepared: the wild type CP expression construct, CPdel-peptide N-terminal fusion construct and CP-peptide C-terminal fusion construct, for both eukaryotic and prokaryotic expression vectors. The wtCP and fusion constructs were generated by PCR amplification of the viral cDNA (pPVA-B11) using appropriate recombinant primers containing PVA CP sequence, a modified epitope from gp100, a melanoma antigen encoding sequence and suitable restriction endonuclease cleavage sites (Table 1).

For preparation of the wtCP constructs, 5′ and 3′ CP oligos (Table 1) were used to amplify the wild type CP gene for cloning into pcDNA and pQE vectors, for eukaryotic and prokaryotic protein expression, respectively. The recombinant CP gene fused to melanoma epitope sequence at the 5′ or at the 3′ end of the CP gene (accordingly N-terminal construct for prokaryotic and eukaryotic expression and C-terminal construct for prokaryotic and eukaryotic expression) was obtained by PCR amplification using appropriate primer pairs (Table 1). In N-terminal fusion protein the first 8 amino acids from the CP N-terminus are substituted with 9 amino acids of the antigenic peptide. The recombinant coat protein bearing the melanoma epitope at the C-terminus (C-terminal fusion protein) has full length.

The sequences encoding wtCP and fusion proteins were amplified by PCR using the high fidelity polymerase Pfu DNA polymerase (Fermentas) according to the manufacturer's instructions. PCR was performed in 50 μl volume: 35 μl H₂O, 5.0 μl 10× Buffer containing MgSO₄ (Fermentas), 4.0 μl 2.5M dNTP, 2.0 μl 20 pmol/μl each primer, 1.0 μl pUFL cDNA as a template, 1.0 μl 2.5 U/μl Pfu polymerase (Fermentas) Amplification conditions: initial denaturation step 94° C. for 1 min, 29 cycles of 20 sec 94° C. (denaturation), 1 min 30 sec 56° C. (annealing) and 2 min 20 sec 72° C. (elongation), 5 min 72° C. to end the synthesis reactions.

PCR products were purified using MoBio UltraClean™ PCR Purification Kit. The purified PCR products and vectors were both digested with restriction enzymes, BamHI (Fermentas) and XhoI (Fermentas) for eukaryotic expression constructs and BamHI and SalI (Fermentas) for prokaryotic expression constructs. Restriction was carried out in 20 μl volume: 50-100 ng vector/100-200 ng PCR product, 4 μl Y⁺/Tango10× Buffer (Fermentas), 0.3 μl 10 U/μl of each restrictase, incubated 1.5 h at 37° C. Restriction products were resolved on 1% agarose gel containing ethidium bromide (0.5 μg/ml) and purified from excised gel slices with MoBio UltraClean™ 15 DNA Purification Kit. The purified DNA fragments were ligated into vectors to generate recombinant expression vectors: 0.3 μl digested and agarose purified vector, 3.9 μl digested and purified recombinant PCR product, 0.5 μl 10× Buffer T4 DNA Ligase (Fermentas), 0.1 μl 10 mM rATP, 0.2 μl 5 U/μl T4 DNA Ligase (Fermentas), incubated 2 h at room temperature (RT).

E. coli Transformation

The competent TG-1 cells were transformed with ligation mixtures. 40 μl of TG-1 cells was added to each ligation mixture, incubated on ice for 45 min, heat shocked 1.5 min at 42° C., 250 μl LB medium was added, incubated 1 h at 37° C. on the eppendorf shaker, cooled on ice, centrifuged 1 min at 7000 rpm, most of the supernatant was removed and cells were plated with the rest of the medium to LB-Ampiccillin (100 μg/ml) plates, grown O/N at 37° C.

Positive clones with recombinant plasmids were screened with colony PCR method and restriction analysis. PCR was conducted in 25 μl reaction: 2.5 μl 10× Taq Buffer (Fermentas), 1.7 μl 25 mM MgCl₂, 2.0 μl 2.5 mM dNTP, 0.6 μl of two PVA specific primers (20 pmol/μl), 16.7 μl mQ, 0.4 μl Taq DNA polymerase (Fermentas), 0.5 μl colony suspended in 2 μl H₂O. Conditions: 94° C. 1 min, 34 cycles of 94° C. 20 sec, 56° C. 1 min 20 sec, 72° C. 1 min, reaction ended with 72° C. 5 min. Samples were analyzed on 1% agarose gel stained with ethidium bromide.

For restriction analysis, plasmid DNA was purified from O/N cultures with Applichem Plas/mini Isolation Spin Kit. Restriction reaction was carried out in 10 μl volume and was analyzed on 1% agarose gel.

For sequencing the recombinant plasmids, reaction was carried out in 10 μl:1 μl BigDye® Terminator Sequencing premix, 2 μl 5× BigDye sequencing buffer, 5 μl H₂O, 1 μl 0.2-0.5 μg/μl plasmid DNA, 1 μl 8 pmol/μl primer (T7 or sp6 primer for pcDNA constructs and pQE Forward or pQE Reverse primer for pQE constructs); conditions: 25 repeats of 20 sec 95° C., 10 sec 50° C., 4 min 60° C. 40 μl of 75% isopropanol was added to reaction mix, vortexed and incubated 5 min at RT, centrifuged 13000 rpm for 10 min. The pellet was washed with 100 μl 70% ethanol, centrifuged 13000 rpm for 7 min, pellet was dried for 5-10 min, resuspended in 15 μl HiDi solution, denatured at 95° C. for 3 min, cooled on ice and sequenced with ABI Prism™ 3130 Genetic Analyzer automat sequenator (Perkin Elmer).

### Mammalian Cell Transfection and Cell Staining

The porcine kidney (PK-15A) cells were seeded into the six well plate at 5×10⁴ cells/per well and grown in Dulbecco's Modified Eagle's Medium (DMEM) supplemented with 50 U/ml penicillin-50 μg/ml streptomycin (PEST) and 10% fetal calf serum (FCS) at 37° C. in CO₂ incubator for 1 h. Cells were transfected with recombinant pcDNA vectors carrying wt PVA CP, N-terminal CP-gp100 fusion or C-terminal CP-gp100 fusion protein sequences, Fugene (Roche Diagnostics) reagent was used for transfection following the purchaser's recommendations and incubated for 24 h (3 μl Fugene and 1 μg plasmid DNA purified with Applichem Plas/mini Isolation Spin Kit was used in this experiment).

The cell-coated slides were washed twice with phosphate buffered saline (PBS) and fixed with a 1 ml mixture of 2% glutaraldehyde and 2% formaldehyde in dH₂O for 15 minutes, then washed twice with PBS, stained with primary antibody (anti-PVA monoclonal antibody A9A4C8 1:500 in PBS) O/N at 4° C. Slides were washed four times with PBST (PBS-0.02% Tween 20), blocked with 0.5% bovine serum albumin (BSA) in PBST 1 h at RT, washed four times with PBST, stained with a secondary anti-mouse Igκ monoclonal antibody coupled to Texas Red (Serotech) (1:2000 in PBS+20 μg/ml BSA). Cells were washed five times with PBST, once with dH₂O, the counter coloration was performed for nuclear staining using Hoechst 33258 dye (Sigma-Aldrich, 1:1000 in dH₂O) for 15 min at RT.

Cells were examined by fluorescence microscopy using an Axiovert 200M invert light microscope (Zeiss) with objective 100× magnification, coloration was observed with “dapi” and “red fluorescence” filters and photographed.

### Protein Expression Evaluation in Prokaryotic Cells

To analyze the ability of the prokaryotic expression clones to express recombinant proteins, rapid screening of small-scale expression cultures was performed. 3 ml LB-Amp was inoculated with 1/50 volume (60 μl) O/N culture and grown at 37° C. until OD₆₀₀=0.5-0.6. 0.5 ml sample was taken to serve as uninduced control. The expression was induced by adding IPTG to a final concentration of 1 mM and the culture was grown 2.5 h at 37° C., 0.5 ml induced culture was taken for control. Both uninduced and induced samples were concentrated by centrifugation at 13000 rpm for 1 min. The pellets of uninduced probes were resuspended in 36 μl PBS and 12 μl 4×SDS sample buffer, pellets from induced probes in 75 μl PBS and 25 μl 4×SDS sample buffer, suspensions were denatured at 95° C. for 3 min. The suspensions were treated back and forth through the syringe with a fine needle, then analyzed on 12% sodium dodecyl sulfate polyacrylamide gel electrophoresis (SDS-PAGE) under conditions 60 mA, 45 min, gels were stained with 0.25% Coomassie brilliant blue R 250 dye (Sigma) and destained with 7% acetic acid.

### Expression of Proteins in Large Scale Cultures

For large scale purification of recombinant proteins, 5 ml of the O/N culture were added into 200 ml LB and incubated at 37° C. until OD600=0.5-0.6. 0.5 ml of uninduced culture was taken as negative control. The cells of large expression culture were induced by IPTG (final concentration 1 mM) and followed by growth for 4 hours at 25° C. for purification under native conditions or at 37° C. for purification under denaturing conditions; 0.5 ml of induced culture was taken for analysis. Probes were treated as described in procedure for protein expression analysis.

The culture was cooled on ice, the cells were pelleted by centrifugation (4000 g, 20 min, 4° C.) and the pellets were stored at −20° C.

### Purification of Recombinant Proteins

Recombinant proteins were purified using the QIAexpress purification system, which is based on the remarkable affinity of nickel-nitrilotriacetic acid (Ni-NTA) resin for the proteins and peptides that contain a 6×His tag—at either N- or C-terminus (Hochuli et al., 1988). The proteins were expressed for large scale purification as described above and used for purification under denaturing or native conditions.

### Purification Procedure Under Denaturing Conditions

The thawed cell pellet was resuspended in 8 ml Buffer B (8M urea, 0.1M NaH₂PO₄, 0.01 M Tris-HCl, pH 8.0, 5 ml/1 g pellet) and mixed gently by shaking at RT for 60 min to lyse the cells. The lysate was centrifuged at 10000 g for 30 min at 4° C. The supernatant (cleared lysate) was taken. 2.5 ml of the Ni-NTA 50% slurry was added to 8 ml cleared lysate and mixed gently by shaking at RT for 1 h. The lysate-Ni-NTA mixture was loaded into a column with the bottom outlet capped. Then the bottom cap was removed and the column flow-through was collected. The column was washed 2 times with 4 ml Buffer C (8M urea, 0.1M NaH₂PO₄, 0.01M Tris-HCl, pH 6.3), and wash fractions were collected for SDS-PAGE analysis. The protein was eluted 4 times with 0.5 ml Buffer D (8M urea, 0.1M NaH₂PO₄, 0.01M Tris-HCl, pH 5.9), 4 times with 0.5 ml Buffer E (8M urea, 0.1M NaH₂PO₄, 0.01M Tris-HCl, pH 4.5), and the eluation fractions were collected for SDS-PAGE analysis.

### Purification Procedure Under Native Conditions

The thawed cell pellet was resuspended in 4 ml lysis buffer (0.05M NaH₂PO₄, 0.3M NaCl, 0.01M imidazole, pH 8.0, 2-5 ml/1 g pellet). Lysis buffer contained 10 mM imidazole to minimize binding of untagged, contaminating proteins and increase purify with fewer wash steps. Then 1 mg/ml lysozyme was added to the lysate and this mixture was incubated on ice for 30 min. The lysate was sonicated at 4° C. using a sonicator (Y3DH-1) equipped with a microtip. Six times 10 sec bursts were used at 5V with a 10 sec cooling period between each burst. The lysate was viscous. The lysate was centrifuged at 10000 g for 30 min at 4° C. to pellet the cellular debris. 30 μl supernatant was taken and stored at −20° C. for SDS-PAGE analysis. The supernatant was saved for purification.

1 ml of the Ni-NTA slurry was added to 4 ml cleared lysate and mixed gently by shaking at 4° C. for 60 min. The lysate-Ni-NTA mixture was loaded into a column with the bottom outlet capped. Then the bottom cap was removed and the column flow-through was collected. The column was washed 2 times with 4 ml wash buffer (0.05M NaH₂PO₄, 0.3M NaCl, 0.02M imidazole, pH 8.0) and wash fractions were collected for SDS-PAGE analysis. Then the protein was eluted 4 times with 0.5 ml eluation buffer (0.05M NaH₂PO₄, 0.3M NaCl, 0.25M imidazole, pH8.0) and the eluation fractions were collected in 4 tubes for SDS-PAGE analysis.

### Dialysis

Dialysis tubings with a cut-off 12000 Da were used (Sigma). It means, that all molecules smaller than 12 kDa would be able to pass freely through the membrane. Protein samples (purified under denaturing conditions) were transferred into tubing, trying to avoid including too many air bubbles. Protein samples were dialyzed gradually as followed. The dialysis tubings containing the protein were inserted into 1 l volume of dialysis buffer (4M urea, 10 mM Tris-HCl, 29.25 g/l NaCl, pH 8.0). The cooled (at 4° C.) mQ water was used for dialysis buffer. The buffer was slowly stirred with a stirbar on magnetic stir plate for 1 h at 4° C. Then 1 l of 100 mM Tris-HCL pH 8.0 was added to the dialysis buffer and protein samples were dialysed for 1 h at 4° C., then 2 l of 50 mM Tris-HCL pH 8.0 was added and dialysed for another hour at 4° C.

Then the protein samples (purified under native and denaturing conditions) were dialyzed against 2 l of 50 mM Tris-HCL, pH 8.0, 250 mM NaCl for 1 h at 4° C. The tubings were removed from the beaker containing dialysis buffer and the dialyzed protein solutions were transferred into tubes and used for immunization of mice.

### Western Blot Analysis

After electrophoresis, proteins were transferred electrophoretically (16V, 9 min) to nitrocellulose membrane (Hybond-C, Amersham, UK) by semi-dry electroblotting. The nitrocellulose membrane was blocked with 3% BSA in TBST (TBS-0.05% Tween 20) solution for 1 h at RT and washed 3 times for 5 min with TBST, incubated O/N at 4° C. with anti-PVA monoclonal antibody (Mab-AP, Adgen) conjugated with enzyme alkaline phosphatase (diluted 1:2500 in TBST). The membrane was washed 3 times for 5 min with TBST solution and 1 time for 5 min with 1×TBS buffer. The enzyme-substrate reaction was developed with NBT/BCIP Tablets (nitro blue tetrazolium chloride/5-bromo-4-chloro-3-indolyl-phophosphate, toluidine-salt) (Boehringer Mannheim GmbH). One tablet was dissolved in distilled 10 ml water, reaction was stopped by adding dH₂O.

### Electron Microscopy

The recombinant proteins were expressed in prokaryotic system in 3 ml LB as described in expression analysis procedure. 1.5 ml cells was harvested by centrifugation at 5000 rpm for 7 min, pellets were resuspended in 100 μl Tris-EDTA buffer (TE) pH 7.5 supplemented with 1 mg/ml lysozyme. The probes were incubated on ice for 30 min and cells were lysed with freezing-melting. 10 μg/ml RNase was added to the cells and incubated on ice for 15 min, centrifuged 13000 rpm for 10 min, supernatant was used for electronmicroscopy. Dilutions of E. coli lysate and/or purified and dialysed proteins were made with TE pH 7.5 (1:10 and 1:100), mounted on carbon-coated grids and incubated 1 h at RT. The grids were washed twice with H₂O, contrasted with 2% uranylacetate for 5 min at RT and dried. The samples were examined using EMV-100BR transmission electron microscope at 30,000× magnification and photographed.

### Mice Immunization

For immunization experiments, C57Bl/6l mice (6-8 weeks old, females) were injected intraperitoneally with appropriate amount of VLPs (50 μg of antigen per mouse), suspended in complete Freund's adjuvant (CFA, Sigma) for the first inoculation and in incomplete Freund's adjuvant (Sigma) for the additional injections.

### Indirect ELISA

To detect antibodies against wt CP or CP-gp 100 fusion proteins in mouse sera, 96-well microtiter Enzyme-Linked ImmunoSorbent Assay (ELISA) plates (Flow) were coated with 1 μg/ml (100 μl/per well) of wtCP/ N-terminal fusion protein/ C-terminal fusion protein diluted in PBS O/N at 4° C. Coated plates were washed 3 times for 5 min with TBST (TBS-0.05% Tween 20), blocked with 1% BSA (in TBST) at RT for 1 h, washed 3 times 5 min with TBST. Mouse sera dilutions (1:20, 1:100, 1:1000, 1:5000, 1:10000 and 1:50000 in TBST) were added to duplicate wells (100 μl/per well) according to coating (protein coating the well corresponded to the protein used for immunization of the mouse originating the sera added to this cell), the plates were incubated O/N at 4° C. The plates were washed 3 times for 5 min with TBST, anti-mouse Horseradish Peroxidase (HRP) at dilution 1:3000 in TBST (100 μl/per well) was added as secondary antibody, incubated for 1 h 40 min at 37° C., washed 3 times for 5 min with TBST and once with TBS. Then 100 μl of the substrate solution (1 mg/ml o-phenylenediamine in 0.1M citrate buffer pH 5.0, containing 0.01% H₂O₂) was added to each cell. Absorbances were measured at wavelength 450 nm after coloration at different timepoints, using a Titertec Multiscan MC Photometer.

To detect antibodies raised against the epitope IMDQVPFSV, synthetic peptide (peptide A191 (NHCOCH₃)CIMDQVPFSV(CONH₂); InbioLabs) covalently conjugated to a carrier protein Keyhole limpet hemocyanin (KLH) was used for coating the wells. During our experiment mouse sera appeared to react with pure KLH, KLH alone was used to coat additional wells. Both KLH-peptide and KLH were diluted to 3 μg/ml in PBS (100 μl/per well). Blocking was performed with 2% skimmed milk powder diluted in TBST. The same mouse sera dilutions were added to duplicate wells coated with both KLH-peptide and to duplicate wells coated with KLH, parallelly. Rest of the analysis was carried out as described above.

### ELISPOT

Enzyme-Linked Immunosorbent Spot (ELISPOT) assay was used to detect low-frequency cytokine producing cells, being more sensitive than conventional ELISA measurements. Interferon-γ (INF-γ) and interleukin-4 (IL-4) producing lymphocytes were detected from spleen cells.

Sterile steps: ELISPOT plates were pre-wet with 100 μl/per well sterile 70% EtOH for 10 min, aspired off, washed twice with sterile PBS, aspired off and flipped dry. Plates were coated with purified anti-mouse INF-γ antibody (BD BioSciences) for INF-γ assay and with purified anti-mouse IL-4 (BD BioSciences) for IL-4 assay, (5 μg/ml in PBS, 100 μl/per well) O/N at 4° C. Plates were washed once with RPMI medium complemented with PEST and 10% FCS (200 μl/per well), blocked with 200 μl/per well RPMI PEST 10% FCS for 2 h at RT. Blocking solution was discarded and 100 μl/per well 10 μg/ml respective antigen (for assay) and Concanavalin A (conA; for positive controls) dilutions 2.5 μg/ml in RPMI with PEST were added to ELISPOT plates. Cell suspensions at different densities (1×, 2× and 4×10⁶ cells/ml) were added to wells (duplicate wells, 100 μl/per well) and duplicate control medium without cells to adjacent wells, covered with lid and incubated in CO₂ incubator for 24 h in INF-γ assay and 48 h in IL-4 assay.

Non-sterile steps: Cell suspension was aspirated, plates were washed twice with dH₂O for 5 min (200 μl/per well), three times with PBST (PBS-0.05% Tween 20). Detection antibody (biotinylated anti-mouse INF-γ for INF-γ assay and biotinylated anti-mouse IL-4 for IL-4 assay, both BD BioSciences) was diluted in PBS 10% FBS (final volume 2 μg/ml), added 100 μl/per well, incubated O/N at 4° C. Wells were washed 3 times with PBST, enzyme streptavidin-HRP (BD BioSciences) was diluted 1:100 in PBS 10% FBS, added 100 μl/per well, incubated 1 h at RT. Wells were washed 4 times with PBST, 2 times with PBS. 100 μl of AEC solution was added per well, monitored spot development 5-60 min, stopped reaction with H₂O wash, discarded. Plates were dried O/N at RT in the dark, spots were counted at magnification 30× with light microscope (Zeiss) and plates were stored at 4° C. in the dark.

AEC solution: 333.3 μl AEC stock mix (10 mg AEC (3-amino-9-ethyl-carbazole)/1 ml DMF (N,N dimethylformamide) was mixed with 10 ml 0.1M acetate (148 ml of 0.2M acetic acid/glacial acetic acid mix with 352 ml of 0.2M sodium acetate added to 1 l dH₂O, pH 5.0), filtered 0.45 μm and 5 μl 130% H₂O₂ was added, used immediately.

### Results

### Generation of Recombinant Expression Vectors

In order to evaluate the positions of the CP gene of PVA which are suitable for foreign epitope expression, the modified gp100 epitope (IMDQVPFSV) encoding sequence of the melanoma was linked to both ends of the CP gene.

The wtCP gene with length of 807 nt and fusion genes were amplified from the infectious cDNA clone pPVA-B11 of PVA-B11 isolate (Puurand et al., 1996) using designed recombinant primer pairs (Table 1), compatible for cloning into pQE or pcDNA vectors. We generated two types of fusion genes—linking the sequence coding for the peptide to the 3′ end of the whole CP gene (giving rise to the C-terminal fusion protein construct) or to the 5′ end with a short deletion fragment of 24 nt (giving rise to the N-terminal construct).

The presence and size of the PCR fragments were analysed on 1% agarose gel.

The fusion genes and the wtCP gene were cloned into procaryotic (pQE-30; Qiagen) and eukaryotic (pcDNA 3.0; Invitrogen) expression vectors: purified PCR products and expression vectors were both digested with appropriate restriction enzymes, PCR products were ligated into vectors, and ligation mixture was transformed into E. coli TG-1 cells. Positive clones were screened with colony-PCR method and restriction analysis. The accuracy of generated recombinant plasmids was confirmed by DNA sequencing.

Thus, wtCP and hybrid N- and C-terminal expression constructs were obtained for both prokaryotic and eukaryotic expression systems. wtCP construct comprised only the wild type CP encoding sequence, N- and C-terminal constructs included fusion of CP sequence with sequence for melanoma gp100 modified peptide IMDQVPFSV.

Protein Expression Detection of Transfected pcDNAs in Mammalian Cells

It is well demonstrated that plasmid DNA encoding a foreign protein can be expressed in situ and it is able to induce immune response against this expressed protein and by that against the pathogen from which the protein was derived (Donnelly et al., 1997; Gurunathan et al., 2000). DNA vaccine based on the potyvirus-like particles expressing immunogenic epitopes could elicit a strong immune response (Pokorna et al., 2005). For example a short sequence encoding for the E7 peptide of HPV was linked to the 3′ end of the gene coding for the entire coat protein of the PVA. Chimeric CPs spontaneously assembled into potyvirus VLPs in eukaryotic cells. Vaccination resulted in a significant protection of mice against the formation of tumours induced by TC-1 tumour cells producing the E7 antigen. Furthermore, when the immunization was performed three days after the administration of tumour cells, 37.5% of mice remained tumour-free for 80 days, compared to 0% of control mice, proving effectiveness in the therapeutic setting as well (Pokorna et al., 2005).

To test the ability of the pcDNA constructs generated in this study to express fusion proteins in eukaryotic conditions, porcine kidney cells (PK-15A) were transfected with recombinant wtCP/pcDNA, N-terminal/pcDNA and C-terminal/pcDNA constructs and stained (cell nucleuses were stained blue with Hoechst 33258 dye (Sigma-Aldrich), PVA CPs were detected with anti-PVA antibodies and stained with Texas Red (Serotech)).

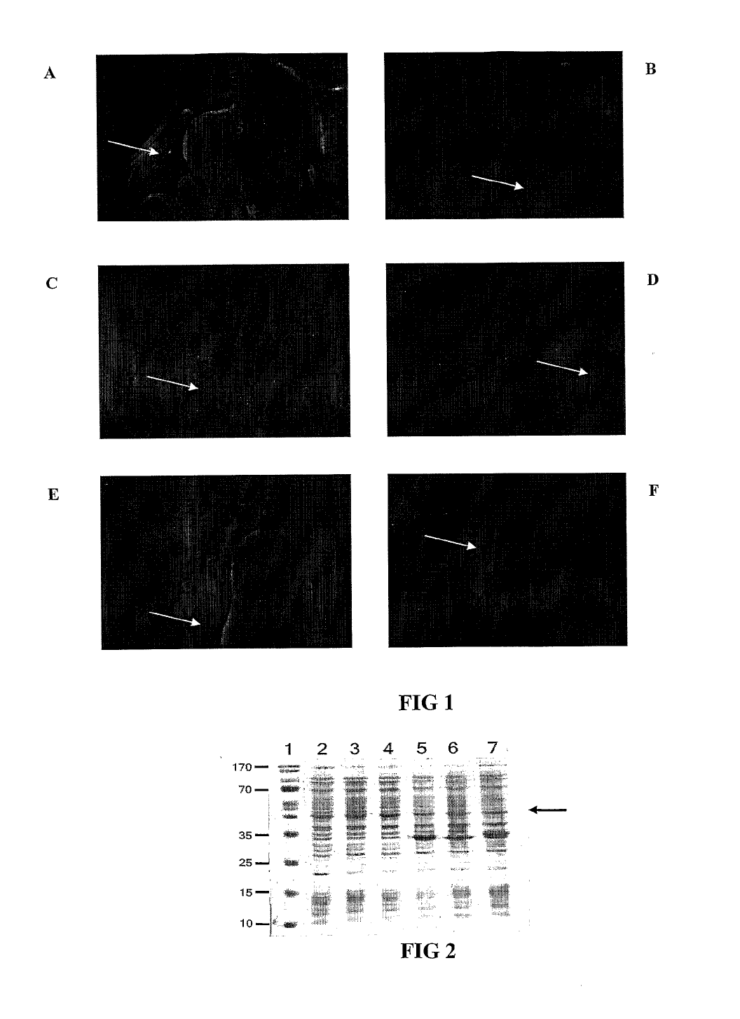

As seen in FIG. 1, all the three recombinant pcDNAs were able to express recombinant PVA CPs, CPs were detected with anti-PVA antibodies and stained with Texas Red. These DNA constructs are planned to be used as DNA vaccines in future study for immunizing mice alone and in combination with purified recombinant CP fusion proteins expressed in E. coli in a later stage of our experiment.

### Expression of Recombinant Proteins in E. Coli and VLP Formation

E. coli TG-1 clones harboring the pQE-30 based recombinant plasmids (wtCP/pQE, CP-gp-Nt/pQE and CP-gp-Ct/pQE constructs) efficiently produced recombinant proteins (6×His tagged wtCP, N- and C-terminal fusion proteins, respectively) expressed in a small volume of E. coli culture after induction with IPTG. The use of small scale expression cultures provides a quick easy way to judge the effects of different growth conditions on expression levels and solubility of recombinant proteins, being therefore very useful before proceeding with a large scale protein expression and purification.

The expression levels of wtCP and fusion proteins in E. coli lysates were high, the correct size and the presence of the expressed proteins was verified on SDS-PAGE (FIG. 2).

One clone of each construct with maximal expression level was chosen and used for large scale expression. Expressed proteins—wtCP and CP fusion proteins—were recognized by anti-PVA monoclonal antibodies (Adgen) in Western blot analysis.

Purification of Recombinant Proteins Expressed in E. coli

Recombinant proteins were purified in preparative amounts by immobilized-metal-affinity chromatography on Ni-NTA resin (Qiagen). Proteins were purified under native and denaturing conditions. In case of purification under denaturing conditions the proteins were eluted from the column in buffer containing 8M urea, 0.1M NaH₂PO₄, 0.01M Tris-HCl, for 4 first elutions the pH of the buffer was 5.9 (buffer D) and for 4 last elutions the pH was 4.5 (buffer E). In case of purification under native conditions the fusion proteins were eluted in buffer containing 0.05M NaH₂PO₄, 0.3M NaCl, 0.25M imidazole, pH 8.0.

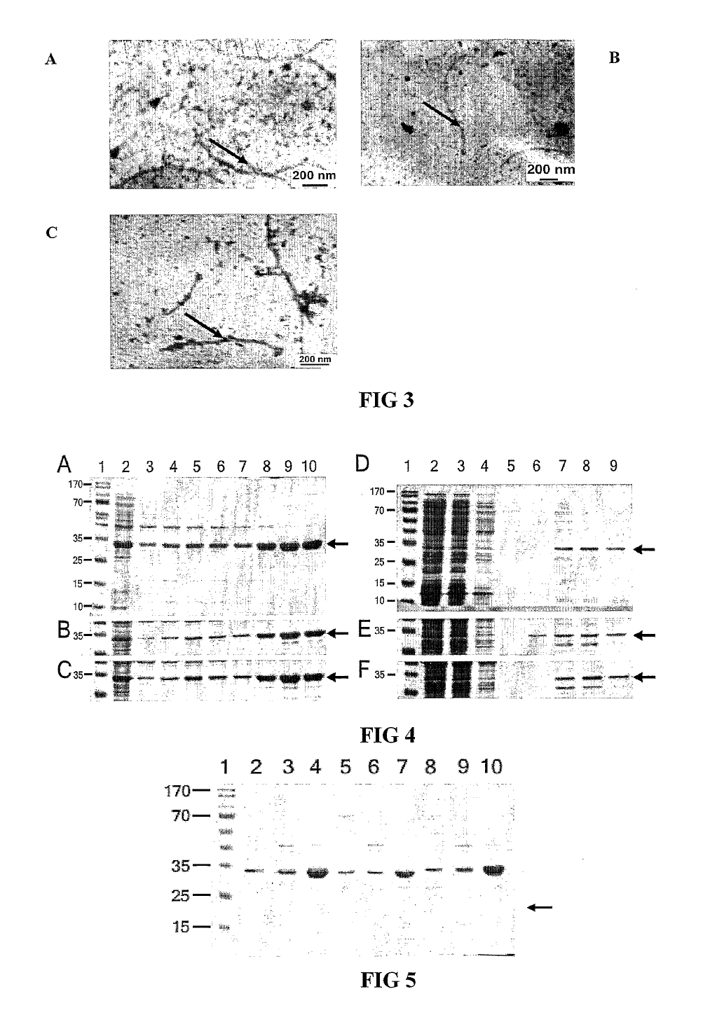

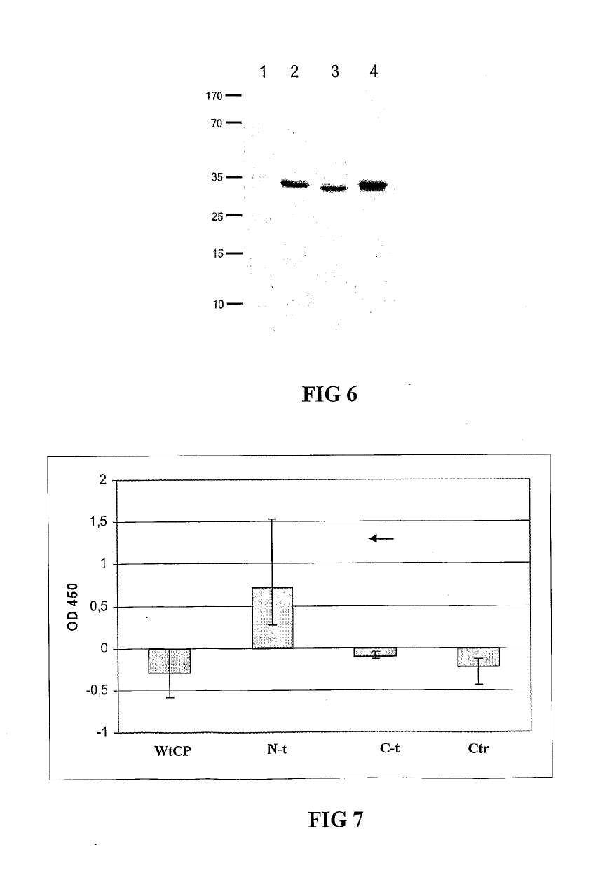

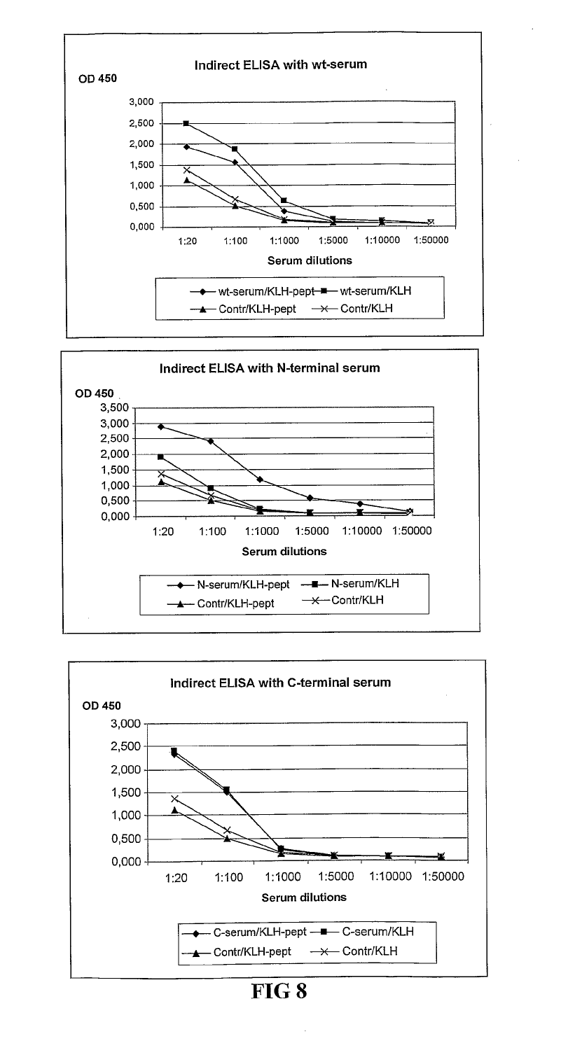

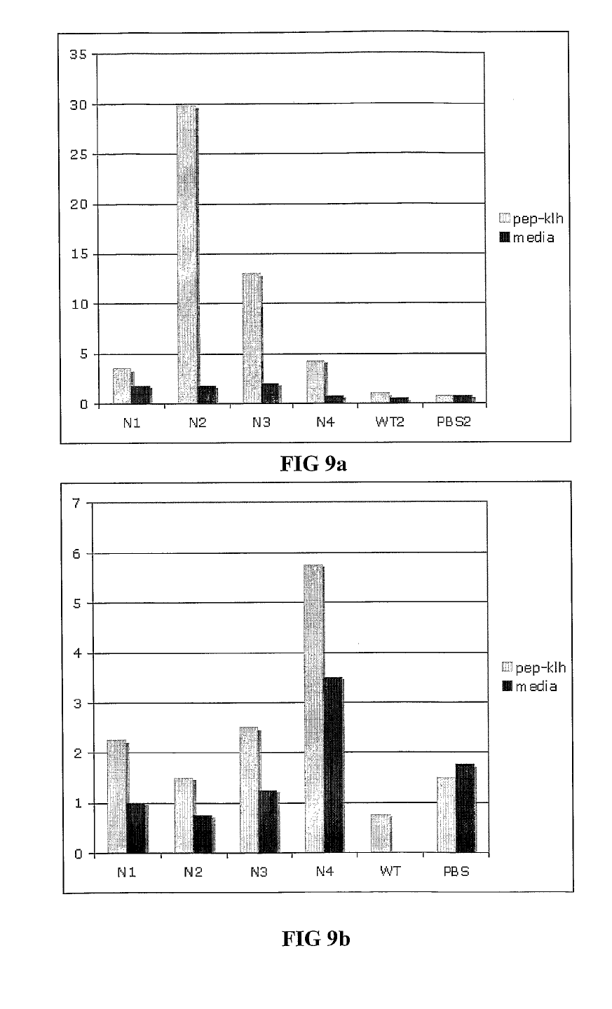

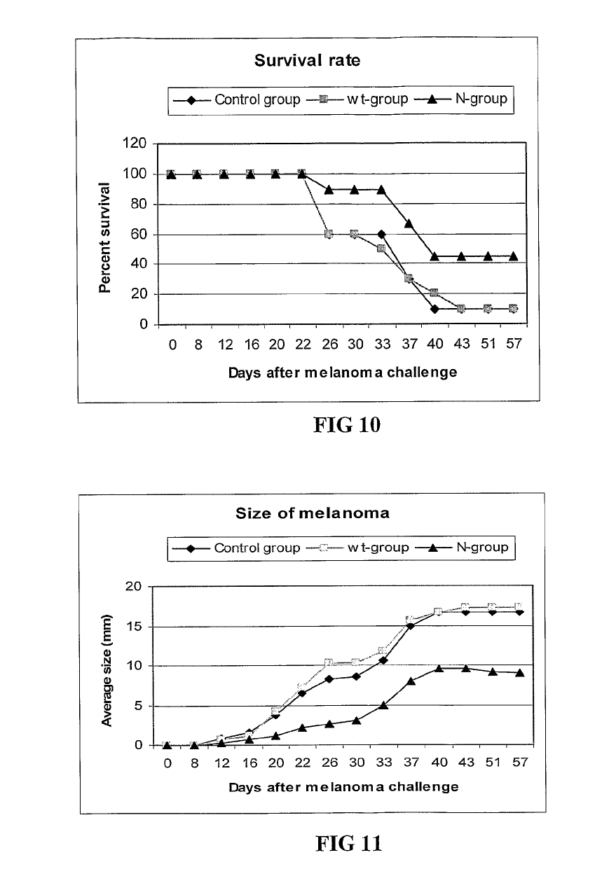

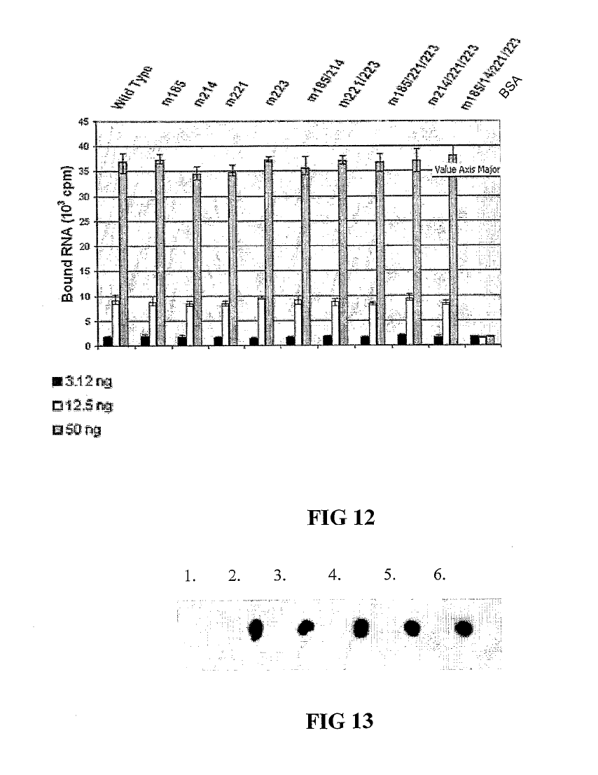

Samples from all elution fractions and control samples were analyzed on 12% SDS-PAGE (FIG. 3).

### Dialysis and Refolding of Recombinant Proteins

Refolding of the solubilized proteins is initiated by the removal of the denaturant. The most used method is the removal of the solubilizing agent by dialysis. During dialysis the concentration of the solubilizing agent decreases slowly, allowing the protein to refold optimally. In our experiment the solubilizing agent used was urea (8M).

Since the elution fractions obtained had quite different concentrations of purified recombinant proteins, we merged in case of native purification elution fractions E2-E4, and in case of denaturing purification, elution fractions E2-E5 into one and E6-E8 into other tube. For proteins purified under denaturing conditions (8M urea) we used stepwise dialysis for urea removing and protein refolding. Thus, urea was removed from the samples by a stepwise decrease of urea (4M, 2M, 1M), followed by dialysis against 50 mM Tris-HCl, 250 mM NaCl, pH8.0 at 4° C., which should be suitable for the refolding of CPs and the reformation of VLPs (Anindya and Savithri, 2003). For proteins purified under native conditions replacement of buffer was performed using dialysis against 50 mM Tris-HCl, 250 mM NaCl, pH8.0 at 4° C.

Purified and dialysed proteins were analysed on 12% SDS-PAGE (FIG. 4).

The obtained proteins were of relatively high purity. High yields of purified proteins were obtained under denaturing conditions (0.5-0.8 mg/100 ml E. coli culture), whereas under native conditions the yields were notably lower (0.05-0.1 mg/100 ml E. coli culture). All the protein solutions were controlled for bacterial endotoxins with The Chromogenic Limulus Amebocyte Lysate Test (Cambrex). All the protein solutions were negative for bacterial endotoxins.

Electron microscopy was used to determine the ability of the expressed recombinant proteins to self-assemble and form VLPs. FIG. 5 shows electron micrographs of purified protein containing the filamentous VLPs formed by wtCPs and by fusion proteins, proving that epitopes can be fused either to the N- or C-terminus of the PVA CP, without abolishing the ability of the protein to polymerize into VLPs when expressed in E. coli, purified and refolded. These results are consistent with the data that N- and C-terminal regions are not involved in the assembly of VLPs (McDonald et al., 1976; Shukla and Ward, 1989; Voloudakis et al., 2004).

To determine the immunogenicity of purified recombinant proteins, denatured proteins were analysed by immunoblot analysis, proteins were recognized by anti-PVA monoclonal antibodies (Adgen) (FIG. 6).

The final concentration of protein purified under native conditions after dialysis was 0.05 mg/ml and the concentration of protein purified under denaturing conditions after dialysis was 0.5 mg/ml. These proteins were used to immunize mice.

Immunization of Mice with Purified Recombinant Proteins

The immunogenicity of recombinant proteins (wtCP, N- and C-terminal proteins) was tested by C57Bl/6l mice immunization. C57Bl/6l mice were immunized intraperitoneally with 50 μg purified wtCP, N- or C-terminal fusion proteins in buffer containing 50 mM Tris-HCl, 250 mM NaCl, pH8.0. The antigen solution was mixed with an equal volume of Freund's adjuvant: for primary immunization the adjuvant was complete Freund's adjuvant. For all other injections incomplete Freund's adjuvant was used. Before the immunization the compatibility of mice line and melanoma cell line was tested. For that, C57Bl/6l mice were injected subcutaneously with 2×10⁶ B16F10 melanoma cells, 8 mice out of 10 developed melanomas, confirming the compatibility of the mice line and melanoma cell line to be used in this study.

Three groups of three C57Bl/6l mice per group were immunized with wtCP, N- or C-terminal fusion proteins, two control mice were immunized with PBS at days 0, 31, 48, 55 and 56. Two days after the last injection blood samples were collected for the detection of antibody response and spleen cells cultivated for ELISPOT assay.

### Antibody Response Against Recombinant Proteins in Mice

The immune response of mice intraperitoneally immunized with purified and refolded wtCP, N- and C-terminal fusion proteins or PBS as control was studied using indirect ELISA analysis.

Antibody titers against purified proteins used to immunize mice were determined using indirect ELISA assay for each serum. The microwell plates were coated with the same recombinant protein against which the serum had been obtained. The plates were blocked with 1% BSA in TBST, the sera were tested in duplicate and in several dilutions (1:20, 1:100, 1:1000, 1:5000, 1:10000, 1:50000). Control serums which were obtained by immunizing mice with PBS were tested against all recombinant proteins. Then anti-mouse HRP was added, absorbance was measured 5 minutes after addition of substrate at wavelength 450 nm (Table 2).

TABLE 2
The results of indirect ELISA against the recombinant
protein used for immunizing the mice.

| | Coating antigen | | |
|---|---|---|---|
| | | N-terminal fusion | C-terminal fusion |
| Serums tested: | WtCP | protein | protein |
| Against CP-fusion | 3.132 | 3.003 | 3.276 |
| proteins | | | |
| Against PBS | 0.264 | 0.187 | 0.135 |

One serum of each immunization group is shown in this table, at dilution 1:100, optical density measured at wavelength 450 nm 5 minutes after substrate adding. Serums were tested against the same recombinant protein against which they had been obtained. Control serums (immunized with PBS) were tested against all recombinant proteins used for immunizing mice.

The results in Table 2 clearly show that all mice immunized with recombinant proteins developed strong antibody responses against these proteins, the responses were up to 1:10000 serum dilution (>2.0 at most serums). The control serums (from mice immunized with PBS) gave significantly lower responses against these fusion proteins (<0.35 at dilution 1:100), indicating that the immune response seen in other serums was not pre-existing in mice. Therefore the recombinant proteins used to immunize mice were highly immunogenic.

The obtained mouse sera against wtCP and fusion proteins are polyclonal and may contain antibodies from different B-cell clones against different antigen epitopes on the surface of CPs.

To determine whether any of these serums contained antibodies raised against the melanoma specific epitope presented on the surface of PVA fusion CPs, serums were analysed in an indirect ELISA assay, where the microwell plates were coated with synthetic peptide CIMDQVPFSV covalently bound to Keyhole limpet hemocyanin (KLH) molecules. The plates were blocked with 2% skimmed milk powder in TBST, the sera were tested in duplicate and in several dilutions (1:20, 1:100, 1:1000, 1:5000, 1:10000, 1:50000). Then anti-mouse HRP was added, absorbance was measured 8 minutes after addition of substrate at wavelength 450 nm. Since during our experiments it turned out that all the serums contained antibodies reacting with the carrier molecule KLH, for all the serums parallel duplicate wells were coated with KLH in PBST.

TABLE 3
The results of indirect ELISA against the synthetic peptide.

| | Responses | Responses | | |
|---|---|---|---|---|
| Serums raised | against | against | Differ- | Difference |
| against: | KLH-peptide | pure KLH | ence* | percentage^{•} |
| wtCP | 1.564 | 1.866 | −0.302 | 19.28% |
| N-terminal protein | 2.411 | 0.855 | 1.526 | 63.29% |
| C-terminal protein | 1.501 | 1.545 | −0.044 | 2.93% |
| PBS | 0.501 | 0.674 | −0.173 | 34.53% |

The *difference is optical density from reaction against KLH-peptide minus optical density from reaction against pure KLH.
^{•}Difference percentage indicates how much the difference is of the response against KLH-peptide.

One serum of each immunization group is shown in this table, at dilution 1:100, optical density measured at wavelength 450 nm 8 minutes after addition of substrate. Since serums reacted with pure KLH, reactions against synthetic peptide bound to KLH and reactions against pure KLH are both shown.

These data in Table 3 clearly demonstrate that the N-terminal fusion protein was able to induce strong antibody response against the presented melanoma specific epitope, showing 63.29% lower reaction rate against pure KLH than against KLH-peptide. In the case of C-terminal fusion protein, wtCP and PBS, reaction against KLH was in every case slightly higher than against KLH-peptide, showing that antibodies against the presented peptide were absent or at most negligibly present.

Because of the technical problems, we were not able to perform ELISPOT assay with the spleen cells cultivated in this immunization test. For this reason, two more mice were immunized with wtCP, three more with N-terminal protein and two more with PBS for control. The amount of the protein in one injection was 50 μg/and the immunization scheme was identical to that performed previously. Moreover, two mice were immunized with wtCP and three more with N-terminal protein according to the immunization scheme used in melanoma testing. Indirect ELISA analysis was performed with all these serums against KLH-peptide and KLH (FIG. 7).

These data in FIG. 7 strongly support our previous conclusions from Table 4 that N-terminal fusion protein is able to induce antibody response against the presented gp100 modified epitope, since only serums raised against the N-terminal protein had higher responses against peptide bound to KLH than against pure KLH (in case of every serum against N-terminal protein), whereas all other serums reacted stronger with pure KLH than KLH-peptide.

This strongly suggests that melanoma specific epitope was relatively better exposed to the immune system when fused to the N-terminus of the PVA CP, compared to fusing the epitope to the C-terminus.

Based on these titration curves (FIG. 8), the dilution 1:100 was chosen for presenting results from indirect ELISA.

### ELISPOT Assay for Detecting Cytokine Producing Cells

VLP vaccines are efficient at stimulating both cellular and humoral immune responses (Schirmbeck et al., 1996; Paliard et al., 2000). ELISPOT assay is a very sensitive way to detect cells producing cytokines, being significantly more sensitive than conventional ELISA measurements. It enables identification and enumeration of cytokine-producing cells at the single cell level, cells are activated with the synthetic peptide of interest. ELISPOT assays produce visual results, each spot that develops in the assay represents a single reactive cell, the product is rapidly captured around the secreting cell.

ELISPOT measuring interleukine-4 (IL-4) secreting cells shows the antibody response against the synthetic peptide that was presented to the immune system in our case on the surface of recombinant CP. Measuring interferone-γ (INF-γ) secreting cells represents cellular immune response against the peptide.

Since the ELISPOT experiment would have been too capacious with cells from all the mice immunized in this second immunization trial, we chose only the mice immunized according to scheme same as in melanoma testing. Mean values of cell counting were calculated, conA as positive control gave noticeably higher cell counting results (>100 in all cases), indicating that the assay worked.

According to FIG. 9a and FIG. 9b, there seems to be a tendency of fusion proteins inducing slightly stronger immune responses (both humoral and cellular) against the synthetic peptide than wtCP or PBS, but since the results are still relatively low and it actually was the first time ELISPOT assay was used in our laboratory, these results are still very preliminary and the assay needs further optimization.

### Testing the Ability of N-Terminal Fusion Protein to Prevent the Development of Melanoma

So far we have demonstrated that the N-terminal fusion protein, unlike C-terminal protein, is able to induce strong antibody response against the melanoma specific epitope presented on the surface of recombinant CP. To determine whether this N-terminal protein could have some effect on the development of melanoma, C57Bl/6l mice were intraperitoneally immunized with recombinant CPs containing 50 μg of wtCP or N-terminal protein in buffer containing 50 mM Tris-HCl, 250 mM NaCl, pH8.0, the antigen was mixed with an equal volume of Freund's adjuvant: for primary immunization complete Freund's adjuvant and for all other injections incomplete Freund's adjuvant was used.

Ten mice were immunized with wtCP and ten mice with N-terminal protein Immunizations were performed at days 0, 7, 14, 28, 29, 30. 6 days later 20 immunized mice (wt-group and N-group) and 10 control mice (control group) received 2×10⁶ B16F10 melanoma cells administered subcutaneously, and the development of melanoma was monitored for a period of 8 weeks. Kinetics of tumour growth was assessed by measuring the tumour diameter, mice were euthanized when an apparent tumour diameter exceeded 1.5 cm. During the immunizations one mouse receiving N-terminal construct died because of an injection error. During the first observation, 8 days after inoculation with melanoma cells, all the mice in all groups were negative for melanoma.

At day 12 first melanomas have already started to develop in all groups: 6 mice in control group (4×1 mm, 2×2 mm), 3 mice in wt-group (2×2 mm, 3 mm) and three mice in N-group (3×1 mm).

At day 16 all groups have increased number of mice with melanomas: 8 mice in control group, 7 mice in wt-group and 6 mice in N-group. In the number of developing melanomas, there seems to be no significant difference among groups, but the medium size of the melanomas varies more: 2.00 mm in control group, 1.57 mm in wt-group and 1.08 mm in N-group. This is the first indication that N-terminal fusion protein might possibly have some negative effect on melanoma development. It should be noted that some mice in all the test groups (3 in control, 2 in wt- and 4 in N-group) lose already-formed melanomas, but in all of these mice, it returns later during the observation, meaning that this regression is most likely irrelevant to our experiment.

At day 26 first mice have developed melanomas requiring the mice to be euthanized. In control group, three mice were euthanized (sizes of melanomas 14, 16, 25 mm), one mouse had died with melanoma of 25 mm and one more mouse had melanoma of the size 1 mm. In wt-group, four mice were euthanized (sizes of melanomas 18, 18, 20, 23 mm) and three more mice had melanomas with sizes 2, 5 and 17 mm. In N-group one mouse had died with 10 mm melanoma, two more mice had melanomas (2 and 7 mm) (Table 4).

TABLE 4
Results of day 26 after challenging mice with melanoma cells.

| | Mice with melanoma | | |
|---|---|---|---|
| | (alive + died/ | Medium size of | Mice died/ |
| | euthanized) | melanomas (mm) | euthanized |
| Control group | 5 | 16.40 | 4 |
| wt-group | 7 | 14.71 | 4 |
| N-group | 4 | 6.67 | 1 |

The total number of mice with melanomas by this day is shown, number of mice who had died or were euthanized by that day and the medium size of all the melanomas in the group by this day.

These results in Table 4 already indicate that immunization of mice with N-terminal fusion proteins might induce immune response, which is able to decelerate the development of melanoma. Although the number of mice with melanomas does not vary significantly, the number of mice who had died or were euthanized is four times higher in control and wt-group than in N-group, and furthermore, the medium size of the melanomas developed is significantly smaller in the N-group (6.67 mm compared to 14.71 or 16.40 mm).

Although in N-group the first mouse died on the day 26 with 10 mm melanoma, the first mouse in this group to develop a melanoma large enough was euthanized on day 37, but in the other two groups the first mice were euthanized on day 26.

By day 43, 9 mice from both control and wt-group had died or were euthanized and one mouse had no melanoma developed (survival rate 10%), in N-group 5 mice had died or were euthanized, three mice were melanoma-free and one had melanoma of the size 5 mm (survival rate 45%) (FIG. 10).

By day 51, all the mice that had not developed melanomas by the day 43, had not developed them by this day too. However, it is noteworthy that the mouse in the N-group, who had 5 mm melanoma, had a regression to 1 mm. If the initial melanoma regressions in all test groups are most likely irrelevant, then this regression is probably the reason of the immunization with N-terminal fusion proteins, since all other melanomas developed this far had resulted in the death of the mice.

By the last day (day 57) of the observation, in the control mice group, who were not immunized and were challenged with melanoma cells, 9 mice had died because of the melanoma and one mouse was disease-free; in wt-group, where mice were immunized with PVA wtCPs and then challenged with melanoma, the final result was identical—nine mice had died because of melanoma and one mouse was disease free; in N-group, where mice were immunized with N-terminal gp100_{209-217(2M)}-CP fusions, five mice had died because of melanoma, three mice were disease free and one mouse who had melanoma with the size of 5 mm in day 43, and the size of 1 mm on day 51, had regressed the melanoma in total (Table 5).

TABLE 5
Results of day 57 after challenging mice with melanoma cells.

| | Mice with melanoma | | |
|---|---|---|---|
| | (alive + died/ | Medium size of | Mice died/ |
| | euthanized) | melanomas (mm) | euthanized |
| Control group | 9 (90%) | 18.56 | 9 |
| wt-group | 9 (90%) | 19.11 | 9 |
| N-group | 5 (55%) | 16.20 | 5 |

The total number of mice with melanomas by day 57 is shown, number of mice who had died or were euthanized by that day and the medium size of all the melanomas in the group by this day (mice who had died earlier were taken into account with the melanoma size of the last measurement, disease-free mice were not included).

According to Table 5, on the last day of observation, day 57, the relative number of mice who had developed melanoma was significantly lower in N-group (55%) than in wt- and control group (90%), and mortality rate was also much lower in N-group than in wt- and control group (55% and 90%, respectively).

As seen on FIG. 10, mice in the N-group had relatively higher survival rates compared to other groups during the entire observation period. Moreover, FIG. 11 demonstrates that the relative size of the melanoma is also significantly smaller, indicating the true effect of this immunization to the regression of melanoma development. This clearly demonstrates the effect of N-terminal CP fusion protein in regressing melanoma development.

To sum up, the results of the experiments do show that both N- and C-terminal PVA CP-gp100_{209-217(2M)} fusion proteins were immunogenic in mice. The antisera against these proteins reacted strongly with the protein against which they had been risen, sera against N-terminal fusion CPs recognized the synthetic peptide IMDQVPFSV, the same sequence that was fused to the recombinant CPs. The control serums gave no reaction with the fusion proteins investigated in this study. N-terminal fusion proteins were able to induce immune response in mice, which was able to delay the development of melanoma and reduce the rate of melanogenic mice.

### Example III

### Development and Characterization of PVA Coat Protein Virus-Like Particles Carrying Melanoma Gp100 Peptide Aa209-217(2M) and/or NY-ESO-1 Peptide

### Example IV

### Therapeutic DNA Vaccination in Combination with Protein Against Tumors

The next step (example IV) is to evaluate the ability of pcDNA constructs as DNA vaccines to induce immune responses in mice, using DNA vaccines alone and in combination with recombinant fusion proteins purified from E. coli.

The therapeutic effect should be evaluated.

1. A potato virus A based vaccine against melanoma.

2. The vaccine according to claim 1 comprising an antigen-presenting system comprising at least one melanoma antigen and a virus-like particle comprising a potato virus A coat protein.

3. The vaccine according to claim 2, wherein mutations selected from the group consisting of R185G, R214G, Q221L and K223L were used for polymerization of potato virus A coat protein subunits into said virus-like particle.

4. The vaccine according to claim 2, comprising an immunogen-carrier complex having an immupotentiation property, said immunogen-carrier complex comprising a virus-like particle carrying at least one immunogen in fusion with a protein or fragment thereof of said virus-like particle for inducing an immune response against melanoma.

5. The vaccine according to claim 2, comprising a protein vaccine comprising a potato virus A coat protein virus-like particles comprising a melanoma antigen.

6. The vaccine according to claim 2, comprising a DNA vaccine comprising a potato virus A coat protein virus-like particles comprising a melanoma antigen.

7. The vaccine according to claim 5, wherein said melanoma antigen is an epitope from gp 100 protein.

8. The vaccine according to claim 7, wherein said melanoma antigen is SEQ ID 11 or SEQ ID 12.

9. The vaccine according to claim 1 for preparation of a medicament for inducing an immune response against a melanoma, which comprises administering to a mammal an effective amount of a composition comprising at least one antigen-presenting system.

10. The vaccine according to claim 1 for preparation of a medicament, wherein the antigen presenting system comprises a DNA vaccine, wherein said antigen-presenting system comprises at least one melanoma antigen.

11. The vaccine according to claim 6, wherein said melanoma antigen is an epitope from gp 100 protein.

12. The vaccine according to claim 11, wherein said melanoma antigen is SEQ ID 11 or SEQ ID 12.

13. The vaccine according to claim 2 for preparation of a medicament for inducing an immune response against a melanoma, which comprises administering to a mammal an effective amount of a composition comprising at least one antigen-presenting system.

14. The vaccine according to claim 2 for preparation of a medicament, wherein the antigen presenting system comprises a DNA vaccine, wherein said antigen-presenting system comprises at least one melanoma antigen.
